(19) United States
(12) Patent Application Publication
(10) Pub. No.: US 2024/0263236 A1
(43) Pub. Date: Aug. 8, 2024

(54) PATHOGENIC GENES ASSOCIATED WITH PREMATURE OVARIAN INSUFFICIENCY AND THEIR APPLICATION

(71) Applicant: SHANDONG UNIVERSITY, Jinan (CN)

(72) Inventors: Zijiang CHEN, Jinan (CN); Yingying QIN, Jinan (CN); Ting GUO, Jinan (CN); Shidou ZHAO, Jinan (CN); Yajuan YANG, Jinan (CN); Xue JIAO, Jinan (CN); Caiyi CHEN, Jinan (CN)

(21) Appl. No.: 18/413,024

(22) Filed: Jan. 15, 2024

Related U.S. Application Data
(63) Continuation of application No. PCT/CN2023/103776, filed on Jun. 29, 2023.

(30) Foreign Application Priority Data
Jan. 17, 2023 (CN) 202310062159.5

(51) Int. Cl. C12Q 1/6883; G16H 50/20

(52) U.S. Cl. CPC C12Q 1/6883; G16H 50/20; C12Q 2600/106; C12Q 2600/154; C12Q 2600/156; C12Q 2600/158

(57) ABSTRACT

The present invention pertains to the realm of disease detection and molecular biology technologies, specifically addressing the pathogenic genes associated with premature ovarian insufficiency and their application. The present invention identified new POI-associated genes through whole-exome sequencing analysis of the POI cohort with the largest international sample size. Their pathogenicity was demonstrated by functional experiments, and mutations of existing known POI-associated genes were further screened. This lays a foundation for the prediction, diagnosis, treatment, genetic etiological analysis and establishment of related genetic models for POI, thus manifesting commendable practical implementation merit.

### CROSS-REFERENCE TO RELATED APPLICATIONS

The present application is a U. S. continuation of co-pending International Patent Application No. PCT/CN2023/103776 filed Jun. 29, 2023, which claims foreign priority of Chinese Patent Application No. 202310062159.5, filed on Jun. 17, 2023 in the State Intellectual Property Office of China, the contents of all of which are hereby incorporated by reference.

### TECHNICAL FIELD

The present invention pertains to the realm of disease detection and molecular biology technologies, specifically addressing the pathogenic genes associated with premature ovarian insufficiency and their application.

### BACKGROUND

The disclosure of information in this part of the background art is only intended to augment comprehension of the overall context of the present invention without necessarily implying acknowledgment or suggesting that such information constitutes prior art commonly known to those skilled in the pertinent art.

Premature ovarian insufficiency (POI), characterized by ovarian dysfunction occurring in women under the age of 40, presents primarily as menstrual irregularities (amenorrhea, oligomenorrhea, or polymenorrhea), elevated gonadotropin levels (FSH>25 U/L), and decreased fluctuating estrogen levels, with an incidence of 3.7% in women under 40. In recent years, factors such as delayed childbearing age, intensified environmental pollution, and prolonged tumor-bearing survival have brought about a noticeable increase in POI incidence. POI not only leads to infertility, but it also tends to be accompanied by systemic multi-system diseases, such as osteoporosis, cardiovascular diseases, psychiatric and neurological conditions, and tumors, compromising the holistic health of women.

The etiology of POI is multifaceted, encompassing genetic factors, autoimmune diseases, infections, environmental factors, and iatrogenic factors. Epidemiological studies indicate a strong genetic predisposition towards POI, with research confirming that daughters have a six-fold increased risk (OR, 95% CI: 6.02, 3.4-10.7) of experiencing early menopause or POI when their mother reached menopause before the age of 45; twin studies also show a high heritability of menopausal age, ranging from 53% to 71%. Thus, genetic factors play a crucial role in the development of POI, encompassing chromosomal abnormalities and gene mutations. Previous Sanger sequencing technologies were inefficient and costly. Recent advancements in next-generation sequencing technologies have remarkably improved the detection efficiency of gene variants and expanded the number of pathogenic genes associated with POI to around 95. However, more than half of patients with POI have unknown etiology, indicating that the contribution of gene variations to POI etiology may be severely underestimated. Detection of POI-related gene variants in the general population or those at high risk of POI is the key to POI prevention and early intervention.

Chinese Patent Literature No. CN202010421306.X disclosed a female POI susceptibility gene detection kit, covering 9 SNP sites of four susceptible genes associated with female POI in Chinese Han women. Chinese Patent Literature 202010351682.6 disclosed a combination of markers and a detection kit for detection of premature ovarian failure genes, screening for a total of 15 SNP sites of susceptibility genes to ovarian premature failure. Chinese Patent Literature No. CN202010729479.8 disclosed a combination of biomarkers for detection of premature ovarian failure genes, comprising 47 SNP sites of 17 genes, along with primers and detection methods targeting such SNP sites.

The POI-related genes included in the above prior art are all known POI pathogenic genes, and they are only detected for specific SNP sites, preventing the discovery of other pathogenic genes and sites in POI patients, which limits the efficiency of disease prediction and diagnosis. Therefore, there remains a deficiency in candidate genes for POI risk prediction and diagnosis at present.

### SUMMARY

In view of the deficiencies of the prior art, the present invention provides pathogenic genes associated with premature ovarian insufficiency and their applications to enable the screening of variant sites associated with the pathogenesis of POI; they can be used to detect, prevent, diagnose or treat POI. The present invention is thus completed based on the aforementioned research findings.

Specifically, the present invention relates to the following technical solutions:

The first aspect of the present invention provides an application of substances for detecting related genes in the preparation of products for detecting premature ovarian insufficiency;

Said relevant genes include ALOX12, BMP6, CPEB1, H1-8, HMMR, HSD17B1, KASH5, LGR4, MCMDC2, MEIOSIN, MST1R, NUP43, PPM1B, PRDM1, RFWD3, SHOC1, SLX4, STRA8, ZAR1, and ZP3.

Further, the substances for detecting the aforementioned relevant genes include, but are not limited to, substances that detect the expression levels, protein modification status (including, but not limited to, methylation, acetylation, phosphorylation, adenylation, ubiquitination, etc.), and mutation status of the genes or proteins encoded by the genes;

Further, the substances for detecting the aforementioned relevant genes specifically include, but are not limited to, substances based on mass spectrometry, DNA microarray, sequencing, allele-specific probe hybridization, restriction fragment analysis, oligonucleotide ligation assays, single-strand conformation polymorphism analysis, and allele-specific amplification assays. Since such substances can be realized by those skilled in the art by appropriate methods based on existing known technologies, they are not further described herein.

Through whole-exome sequencing analysis of the largest international cohort of POI samples, the present invention discovered above-noted 20 new POI pathogenic genes, and verified their pathogenicity through functional experiments. Specifically, by detecting the mutation status of the aforementioned genes, ACMG classification was performed on detected gene mutations to yield pathogenic or suspected pathogenic (P/LP) mutations.

Specifically, the details of said pathogenic genes and their mutation sites are as follows:

| Gene | Gene ID | Mutation site |
|---|---|---|
| PPM1B | 5495 | NM_002706.6: c.1253dup |
| ALOX12 | 239 | NM_000697.3: c.1909G > T |
| LGR4 | 55366 | NM_018490.5: c.2449C > T |
| MCMDC2 | 157777 | NM_173518.5: c.204del |
| ALOX12 | 239 | NM_000697.3: c.542 + 1G > A |
| HSD17B1 | 3292 | NM_001330219.3: c.313G > T |
| BMP6 | 654 | NM_001718.6: c.472C > T |
| SHOC1 | 158401 | NM_173521.5: c.3903G > A |
| CCDC155 | 147872 | NM_144688.5: c.1269 + 2T > A |
| SHOC1 | 158401 | NM_173521.5: c.231_232del |
| CCDC155 | 147872 | NM_144688.5: c.218del |
| HMMR | 3161 | NM_001142556.2: c.1386-2A > C |
| RFWD3 | 55159 | NM_001370535.1: c.789del |
| STRA8 | 346673 | NM_182489.1: c.258 + 1G > A |
| LGR4 | 55366 | NM_018490.5: c.903-3_903-2del |
| PRDM1 | 639 | NM_001198.4: c.1866C > G |
| MST1R | 4486 | NM_002447.4: c.1480_1481del |
| ALOX12 | 239 | NM_000697.3: c.805C > T |
| HSD17B1 | 3292 | NM_001330219.3: c.721-1G > C |
| HSD17B1 | 3292 | NM_001330219.3: c.947dup |
| MST1R | 4486 | NM_002447.4: c.3846del |
| NUP43 | 348995 | NM_198887.3: c.641del |
| ZAR1 | 326340 | NM_175619.3: c.1131 + 2T > C |
| SLX4 | 84464 | NM_032444.4: c.100C > T |
| RFWD3 | 55159 | NM_001370535.1: c.1561_1562del |
| SHOC1 | 158401 | NM_173521.5: c.1939 + 2T > A |
| HMMR | 3161 | NM_001142556.2: c.667G > T |
| ZP3 | 7784 | NM_001110354.2: c.52G > T |
| HSD17B1 | 3292 | NM_001330219.3: c.313G > T |
| ZP3 | 7784 | NM_001110354.2: c.1028G > A |
| CPEB1 | 64506 | NM_001365240.1: c.1438_1444del |
| ZP3 | 7784 | NM_001110354.2: c.633_652del |
| SHOC1 | 158401 | NM_173521.5: c.3101_3102dup |
| SLX4 | 84464 | NM_032444.4: c.4390_4393dup |
| ZAR1 | 326340 | NM_175619.3: c.119G > A |
| MEIOSIN | 388553 | NM_001310124.2: c.654del |
| HSD17B1 | 3292 | NM_001330219.3: c.97 + 1G > A |
| CPEB1 | 64506 | NM_001365240.1: c.1140_1144 + 10del |
| ZAR1 | 326340 | NM_175619.3: c.270_297dup |
| ZAR1 | 326340 | NM_175619.3: c.44dup |
| H1FOO | 132243 | NM_153833.2: c.251_261del |
| SLX4 | 84464 | NM_032444.4: c.928C > T |
| ZAR1 | 326340 | NM_175619.3: c.1132-2A > C |
| H1FOO | 132243 | NM_153833.2: c.212del |
| NUP43 | 348995 | NM_198887.3: c.544C > T |
| MCMDC2 | 157777 | NM_173518.5: c.685C > T |
| ZAR1 | 326340 | NM_175619.3: c.270_297dup |
| ZAR1 | 326340 | NM_175619.3: c.1108C > T |
| HMMR | 3161 | NM_001142556.2: c.145 + 1G > A |
| MST1R | 4486 | NM_002447.4: c.3991C > T |
| CCDC155 | 147872 | NM_144688.5: c.876 + 1G > A |
| PPM1B | 5495 | NM_002706.6: c.644_671del |
| PRDM1 | 639 | NM_001198.4: c.2322_2325dup |
| SHOC1 | 158401 | NM_173521.5: c.231_232del |
| BMP6 | 654 | NM_001718.6: c.1141C > T |
| HMMR | 3161 | NM_001142556.2: c.429del |
| MEIOSIN | 388553 | NM_001310124.2: c.214A > T |
| LGR4 | 55366 | NM_018490.5: c.1580-2dup |
| PRDM1 | 639 | NM_001198.4: c.32del |
| MST1R | 4486 | NM_002447.4: c.1230 + 2T > C |
| SHOC1 | 158401 | NM_173521.5: c.143_146del |
| NUP43 | 348995 | NM_198887.3: c.639del |

The (etiology) screening, (auxiliary) diagnosis, detection, monitoring and predictive evaluation of premature ovarian insufficiency are realized by detecting above-noted pathogenic genes and their mutations. Therefore, said product is one that can be used for aforementioned (etiology) screening, (auxiliary) diagnosis, detection, monitoring and predictive evaluation of premature ovarian insufficiency.

Further, through research, the present invention identified new P/LP mutation sites within known POI pathogenic genes, which also mediate the occurrence of POI disease. The details are as follows:

| Gene | Gene ID | Mutation site |
|---|---|---|
| GDF9 | 2661 | NM_005260.5: c.612del |
| AARS2 | 57505 | NM_020745.4: c.1360G > T |
| BLM | 641 | NM_001287246.2: c.1949C > T |
| RECQL4 | 9401 | NM_004260.3: c.3034T > C |
| MCM8 | 84515 | NM_001281521.1: c.1553G > A |
| BMP15 | 9210 | NM_005448.2: c.373C > T |
| PMM2 | 5373 | NM_000303.3: c.526G > A |
| FIGLA | 344018 | NM_001004311.3: c.385-2A > G |
| SPIDR | 23514 | NM_001080394.4: c.2102del |
| RECQL4 | 9401 | NM_004260.3: c.2886-1G > A |
| HFM1 | 164045 | NM_001017975.5: c.3698C > T |
| WRN | 7486 | NM_000553.6: c.1270-1G > A |
| RECQL4 | 9401 | NM_004260.3: c.3515A > G |
| MSH4 | 4438 | NM_002440.4: c.911dup |
| HFM1 | 164045 | NM_001017975.5: c.2336A > C |
| GDF9 | 2661 | NM_005260.5: c.333C > G |
| LMNA | 4000 | NM_170707.4: c.991C > T |
| SPIDR | 23514 | NM_001080394.4: c.2209C > T |
| HFM1 | 164045 | NM_001017975.5: c.1792G > C |
| HFM1 | 164045 | NM_001017975.5: c.3784G > A |
| FIGLA | 344018 | NM_001004311.3: c.385-2A > G |
| HSF2BP | 11077 | NM_007031.2: c.291 + 1G > T |
| MRPS22 | 56945 | NM_020191.4: c.1043_1046dup |
| HARS2 | 23438 | NM_001363535.2: c.567del |
| BLM | 641 | NM_001287246.2: c.2980del |
| NR5A1 | 2516 | NM_004959.5: c.1262dup |
| SGO2 | 151246 | NM_152524.6: c.195dup |
| MCM8 | 84515 | NM_001281521.1: c.1857G > A |
| MCM9 | 254394 | NM_017696.2: c.398C > G |
| C14orf39 | 317761 | NM_174978.3: c.1037C > G |
| POF1B | 79983 | NM_001307940.2: c.1438-1G > T |
| BRCA2 | 675 | NM_000059.3: c.7084_7085del |
| C14orf39 | 317761 | NM_174978.3: c.1349dup |
| RECQL4 | 9401 | NM_004260.3: c.2128C > T |
| MCM9 | 254394 | NM_017696.2: c.78_80del |
| MCM9 | 254394 | NM_017696.2: c.1390G > A |
| MCM9 | 254394 | NM_017696.2: c.398C > G |
| MCM9 | 254394 | NM_017696.2: c.1306A > G |
| RECQL4 | 9401 | NM_004260.3: c.2404G > T |
| RECQL4 | 9401 | NM_004260.3: c.2554_2559dup |
| WDR62 | 284403 | NM_001083961.2: c.3203_3206del |
| HFM1 | 164045 | NM_001017975.5: c.3477A > C |
| WDR62 | 284403 | NM_001083961.2: c.178-2A > G |
| COX10 | 1352 | NM_001303.4: c.954_955del |
| HARS2 | 23438 | NM_001363535.2: c.1171_1183del |
| HFM1 | 164045 | NM_001017975.5: c.2028_2029del |
| AARS2 | 57505 | NM_020745.4: c.985del |
| NUP107 | 57122 | NM_020401.4: c.262C > T |
| AARS2 | 57505 | NM_020745.4: c.2005C > T |
| SPIDR | 23514 | NM_001080394.4: c.2656_2660del |
| PSMC3IP | 29893 | NM_016556.4: c.597 + 1G > T |
| FIGLA | 344018 | NM_001004311.3: c.1A > G |
| NR5A1 | 2516 | NM_004959.5: c.821T > C |
| BLM | 641 | NM_001287246.2: c.2310C > G |
| HFM1 | 164045 | NM_001017975.5: c.1730A > G |
| SPIDR | 23514 | NM_001080394.4: c.1174G > T |
| AIRE | 326 | NM_000383.4: c.993del |
| HSF2BP | 11077 | NM_007031.2: c.973G > T |
| BMP15 | 9210 | NM_005448.2: c.542G > A |
| EXO1 | 9156 | NM_130398.4: c.353_354del |
| SPIDR | 23514 | NM_001080394.4: c.1181C > G |
| HFM1 | 164045 | NM_001017975.5: c.3934C > T |
| RECQL4 | 9401 | NM_004260.3: c.3418del |
| BRCA2 | 675 | NM_000059.3: c.1910-1G > A |
| RECQL4 | 9401 | NM_004260.3: c.1918C > G |
| MCM8 | 84515 | NM_001281521.1: c.1111_1112del |
| WDR62 | 284403 | NM_001083961.2: c.1255C > T |
| GALT | 2592 | NM_000155.4: c.551del |
| MCM9 | 254394 | NM_017696.2: c.1151-1G > A |
| MCM9 | 254394 | NM_017696.2: c.322G > T |
| RECQL4 | 9401 | NM_004260.3: c.2233C > T |
| AARS2 | 57505 | NM_020745.4: c.298C > T |
| MCM9 | 254394 | NM_017696.2: c.1390G > A |
| POF1B | 79983 | NM_001307940.2: c.137dup |
| MSH4 | 4438 | NM_002440.4: c.2546A > T |
| CLPP | 8192 | NM_006012.4: c.6G > A |
| STAR | 6770 | NM_000349.3: c.403G > T |
| FANCM | 57697 | NM_020937.4: c.5653C > T |
| GNAS | 2778 | NM_080425.3: c.344G > A |
| FOXL2 | 668 | NM_023067.4: c.427dup |
| TP63 | 8626 | NM_003722.5: c.1703del |
| AIRE | 326 | NM_000383.4: c.1182C > A |
| PMM2 | 5373 | NM_000303.3: c.492del |
| RECQL4 | 9401 | NM_004260.3: c.2288G > A |
| SPATA22 | 84690 | NM_001321337.1: c.400C > T |
| POLG | 5428 | NM_001126131.2: c.1168del |
| SPIDR | 23514 | NM_001080394.4: c.3G > A |
| MSH4 | 4438 | NM_002440.4: c.1345T > G |
| MSH4 | 4438 | NM_002440.4: c.1094T > A |
| AIRE | 326 | NM_000383.4: c.44G > A |
| FANCM | 57697 | NM_020937.4: c.1213C > T |
| ATM | 472 | NM_000051.3: c.5830del |
| BLM | 641 | NM_001287246.2: c.2693G > A |
| BLM | 641 | NM_001287246.2: c.1019A > G |
| HFM1 | 164045 | NM_001017975.5: c.1130A > G |
| BNC1 | 646 | NM_001717.4: c.2476C > T |
| PMM2 | 5373 | NM_000303.3: c.448-2A > G |
| NR5A1 | 2516 | NM_004959.5: c.292A > T |
| STAG3 | 10734 | NM_001282717.1: c.2938del |
| HARS2 | 23438 | NM_001363535.2: c.1171_1183del |
| MCM9 | 254394 | NM_017696.2: c.3160A > C |
| HFM1 | 164045 | NM_001017975.5: c.1153A > G |
| MSH4 | 4438 | NM_002440.4: c.1603C > T |
| MCM8 | 84515 | NM_001281521.1: c.1385C > A |
| MRPS22 | 56945 | NM_020191.4: c.1043_1046dup |
| MCM9 | 254394 | NM_017696.2: c.816T > G |
| NR5A1 | 2516 | NM_004959.5: c.245-2A > G |
| FSHR | 2492 | NM_000145.4: c.447-1G > C |
| HARS2 | 23438 | NM_001363535.2: c.567del |
| CLPP | 8192 | NM_006012.4: c.368-3_368-2dup |
| MSH4 | 4438 | NM_002440.4: c.2179G > C |
| WRN | 7486 | NM_000553.6: c.−76-1G > A |
| SYCP2L | 221711 | NM_001040274.3: c.1219-1G > C |
| POLG | 5428 | NM_001126131.2: c.2677T > C |
| STAR | 6770 | NM_000349.3: c.125del |
| RECQL4 | 9401 | NM_004260.3: c.3062_3079dup |
| FANCL | 55120 | NM_001114636.1: c.555 + 2T > G |
| MCM9 | 254394 | NM_017696.2: c.595T > G |
| ERCC6 | 2074 | NM_000124.4: c.1821 + 1G > T |
| NUP107 | 57122 | NM_020401.4: c.8 + 1G > C |

Notably, this application also discovered for the first time that TP63 gene mutation sites c.1928G>A, c.1937T>C, and c.1964G>A mediated the occurrence of POI. Therefore, TP63 genes carrying mutation sites c.1928G>A, c.1937T>C, c.1964G>A, or TP63 proteins carrying protein mutation sites p.R643Q, p.L646P, p.R655Q can serve as biomarkers for (etiology) screening, (auxiliary) diagnosis, detection, monitoring and predictive evaluation of premature ovarian insufficiency. Hence, they are also within the scope of protection of this application.

In practice, said product may exist in any known form such as primers, probes, nucleic acid membrane strips, (gene or protein) chips, preparations, kits, instruments, detection devices and equipment. Since those skilled in the art can implement the aforementioned products without creative labor under actual circumstances, all such products fall within the scope of protection of the present application.

The second aspect of the present invention provides a product for detecting premature ovarian insufficiency, which contains substances for detecting related genes.

Since the substances for detecting relevant genes are the same as those in the first aspect, they are not further described here.

In conclusion, the (etiology) screening, (auxiliary) diagnosis, detection, monitoring and predictive evaluation of premature ovarian insufficiency are realized by detecting above-noted pathogenic genes and their mutations. Therefore, said product is one that can be used for aforementioned (etiology) screening, (auxiliary) diagnosis, detection, monitoring and predictive evaluation of premature ovarian insufficiency.

Said product may exist in any known form such as primers, probes, nucleic acid membrane strips, (gene or protein) chips, preparations, kits, instruments, detection devices and equipment. Since those skilled in the art can implement the aforementioned products without creative labor under actual circumstances, all such products fall within the scope of protection of the present application.

The third aspect of the present invention provides a system for detecting premature ovarian insufficiency, which comprises:
i) An analysis unit, which comprises: Substances chosen from above-noted related genes in the subject's sample to be tested, and;
ii) An assessment unit, which comprises: Judging the conditions of the subject based on the profile of the relevant genes identified in i).

Wherein, the test sample may be a human sample, more specifically, said test sample comprises peripheral blood from the subject.

Since the detection substances for relevant genes are the same as those in the above-noted first aspect, they are not further described here.

Said subject's disease profile includes screening, (auxiliary) diagnosis, detection, monitoring, and predictive evaluation of premature ovarian insufficiency in the subject.

The fourth aspect of the present invention provides an application of the above-noted relevant genes in preparing drugs for premature ovarian insufficiency and/or screening drugs for premature ovarian insufficiency.

Said drugs for premature ovarian insufficiency refer to the ones that prevent and/or treat premature ovarian insufficiency.

Said drugs may also include pharmaceutically acceptable carriers. Said pharmaceutically acceptable carriers may be buffers, emulsifiers, suspension agents, stabilizers, preservatives, excipients, fillers, coagulants and blending agents, surfactants, diffusants or defoamers.

Said drugs may include pharmaceutically acceptable carriers. Said pharmaceutically acceptable carriers may be viruses, microcapsules, liposomes, nanoparticles or any combination thereof. The delivery agents of said pharmaceutically acceptable carriers may be liposomes, biocompatible polymers, lipoproteins, peptides, polysaccharides, lipopolysaccharides, artificial viral envelopes, inorganic particles, as well as bacteria or viruses, phages, lectins or plasmid carriers.

Said drugs may also be used in combination with other drugs to prevent and/or treat premature ovarian insufficiency, and other preventive and/or therapeutic compounds may be administered simultaneously with the primary active ingredients, or even in the same composition.

Said drugs may also be administered singly as separate formulations or differing dosage forms from its primary active ingredients along with additional preventative and/or treatment components. Part of the primary ingredients' dosage may be administered concurrently with other therapeutic compounds, while the remaining doses can be administered individually. The dose of the drug of the present invention can be adjusted depending on the severity of symptoms, the frequency of recurrence and the physiological response of the treatment regimen during the treatment.

The drug of the present invention can be administered into the body by a known means. For instance, it could be conveyed systemically through veins or injected locally into the desired tissues. Optionally, administration can occur by intravenous, transdermal, nasal, mucosal, or other delivery methods. Such administration can take place via single-dose or multi-dose regimens. It is understood by those skilled in the art that the actual dose to be administered in the present invention may vary largely depending on a number of factors, such as target cells, biological type or its tissues, general condition of the subject to be treated, route of administration, and method of administration, and so on.

Beneficial technical effects of one or more of above-noted technical solutions:

The technical solutions above identified new POI-associated genes through whole-exome sequencing analysis of the POI cohort with the largest international sample size. Their pathogenicity was demonstrated by functional experiments, and mutations of existing known POI-associated genes were further screened. This lays a foundation for the prediction, diagnosis, treatment, genetic etiological analysis and establishment of related genetic models for POI, thus manifesting commendable practical implementation merit.

### BRIEF DESCRIPTION OF DRAWINGS

The drawings attached to the specification forming part of the invention are used to provide an enhanced comprehension of the present invention. Schematic embodiments of the invention and their descriptions are used to interpret the invention without posing undue restrictions upon its breadth.

FIG. 1 shows the results of functional verification for mutations in BLM, HFM1, MSH4, NR5A1, MCM8, MCM9 and RECQL4 genes in Embodiment 1 of the present invention.

FIGS. 2A to 2K are functional verification tests for PRDM1, STRA8 and MCMDC2 gene mutations in Embodiment 1 of the present invention, wherein “FIG. 2A” is a pattern map of the PRDM1 gene and the location of mutation; “FIG. 2B” is the western blot result of overexpressing wild-type and mutant PRDM1 proteins in vitro; “FIG. 2C” is the immunofluorescence result of wild-type and mutant protein, reflecting their localization in cells; “FIG. 2D” is the analysis of degradation of wild-type and mutant proteins after CHX treatment; “FIG. 2E” is the pattern map of the STRA8 gene and mutations; “FIGS. 2F-2G” are the results of Minigene validation for STRA8 mutation; “FIG. 2H” is the result of immunofluorescence staining of wild-type and mutant STRA8 proteins, reflecting their localization in cells; “FIG. 2I” is the pattern map of the MCMDC2 gene and mutations; “FIG. 2J” is the schematic diagram of the GFP reporter system; and “FIG. 2K” is the flow report diagram and statistical analysis diagram of the detection of HR repair efficiency of wild-type and mutant MCMDC2 proteins.

FIGS. 3A and 3B show the 20 newly identified POI-associated genes in Embodiment 1 of the present invention, wherein “FIG. 3A” shows the 32 genes with significant differences in the case-control analysis, of which 12 are known genes and 20 are newly identified associated genes; “FIG. 3B” illustrates the follicular development process where the 20 newly associated genes for POI are located.

FIGS. 4A and 4B show the Sanger sequencing validation results of TP63 mutations in POI patients in Embodiment 2 of the present invention, wherein, “FIG. 4A” illustrates the Sanger sequencing results of nine TP63 mutations in POI patients, while “FIG. 4B” shows the sequencing results of mutations in POI patient No. 6 and its parents.

FIGS. 5A and 5B are pattern maps showing the location of the TAp63α mutation site in Embodiment 2 of the present invention, wherein, “FIG. 5A” is a pattern map of the structure of TAp63α protein and its mutation sites, while “FIG. 5B” shows the species conservation analysis of the four mutation sites located in core region of the TID.

FIGS. 6A, 6B and 6C illustrate the experimental result of TAp63α mutation disrupting protein homeostasis in Embodiment 2 of the present invention, where, “FIG. 6A” shows the detection result of wild-type and mutant TAp63α proteins; “FIG. 6B” shows the detection result of the conformation of wild-type and mutant TAp63α proteins; and “FIG. 6C” indicates the detection result of interaction of the TAp63αΔTID with WT or mutant TID.

FIGS. 7A, 7B, 7C, 7D and 7E illustrate the experimental result of mutant TAp63α inducing apoptosis in Embodiment 2 of the present invention, wherein, “FIG. 7A” is the result of luciferase reporter gene assay; “FIGS. 7B and 7C” are the mutant TAp63α resulting in increased BAX protein expression; and “FIGS. 7D and 7E” are the outcome of TUNEL assay.

FIGS. 8A, 8B, 8C and 8D show the construction strategy and phenotypic characterization results of p63^{+/ΔTID} mice in Embodiment 2 of the present invention, wherein, “FIGS. 8A and 8B” show the construction strategy and genotypic characterization of p63^{+/ΔTID} mice; “FIG. 8C” shows the results of p63 protein detection in the ovaries of p63^{+/ΔTID} mice; and “FIG. 8D” shows the results of phenotypic and fertility test of p63^{+/ΔTID} mice.

FIGS. 9A, 9B and 9C present the POI-like phenotype result of p63^{+/ΔTID} female mice in Embodiment 2 of the present invention, wherein, “FIGS. 9A and 9B” show the result of premature follicle depletion in the ovaries of p63^{+/ΔTID} female mice, while “FIG. 9C” illustrates the result of premature oocyte loss in the ovaries of p63^{+/ΔTID} female mice.

FIGS. 10A, 10B and 10C present the results of spontaneous apoptosis occurring in p63^{+/ΔTID} mice oocytes in Embodiment 2 of the present invention, wherein, “FIG. 10A” is the result of immunofluorescence staining of Cleaved-PARP1, and “FIGS. 10B and 10C” are the result of expression detection of apoptosis-related target genes downstream of p63.

### DESCRIPTION OF EMBODIMENTS

It should be noted that the following detailed descriptions are all illustrative and intended to provide further clarification of the present application. Unless otherwise indicated, all technical and scientific terms used herein have the same meaning as commonly understood by one of ordinary skill in the art to which this application pertains.

It is important to note that the terms used here are intended only to describe specific embodiments and are not intended to limit exemplary embodiments under the present application. As used here, the singular form is also intended to include the plural form, unless the context expressly states otherwise. It should also be understood that when the terms “comprise” and/or “include” are used in this specification, they indicate the presence of features, steps, operations, devices, components, and/or combinations thereof. Experimental methods in the following specific embodiments in which specific conditions are not indicated are generally in line with conventional methods and conditions of molecular biology within the art of the field, such technologies and conditions being fully explained in the literature. Refer to, for example, the technologies and conditions described in Molecular Cloning: a Laboratory Manual by Sambrook et al., or follow the conditions recommended by the manufacturer.

The present invention is further described in connection with specific embodiments, and the following examples are for the purpose of explaining the invention only and are not intended to limit its contents. If the specific conditions of the experiment are not indicated in the embodiments, they are usually in accordance with the conventional conditions, or in accordance with the conditions recommended by the sales company; the materials, reagents, etc., used in the embodiments are commercially available, unless otherwise specified.

The following is a further explanatory description of the present invention by way of embodiments, but it does not constitute a limitation on the present invention. It should be noted that these embodiments are used to describe the present invention instead of limiting its scope.

### Embodiment 1

Through whole-exome sequencing analysis of the largest international cohort of POI samples, the present embodiment discovered 20 new POI pathogenic genes, and verified their pathogenicity through functional experiments.

### 1. Inclusion of POI Patients:

The POI cohort of this embodiment included a total of 1030 patients with POI, excluding known causes of POI, such as chromosomal abnormalities, autoimmune diseases, and medical factors such as ovarian surgery or radiotherapy; it's the idiopathic POI cohort with the largest sample size in the world to date.

### 2. Control Inclusion:

Whole exome data of 5000 cases of normal population from the database were used.

### 3. Whole Exome Sequencing and Data Analysis:

### (1) DNA Sample Preparation and Exome Sequencing

We performed whole exome sequencing on all 1030 POI patients and extracted genomic DNA from EDTA-anticoagulated peripheral blood for all samples using the QIAamp DNA Blood Mini Kit (Qiagen), and took 1 ug for library preparation. DNA libraries were prepared using AIExome Enrichment Kit V1 (iGeneTech, Beijing, China), and 150 bp-200 bp-long DNA fragments were sequenced using NovaSeq platform (Illumina, San Diego, CA). Reads were subsequently aligned to the human genome reference sequence GRCh37/hg19 using Burrows-Wheeler Aligner MEM. Duplicate Reads were removed and single nucleotide variant SNVs and minor deletion (del) were identified using Genome Analysis Toolkit (GATK). The variants screened were annotated using the Ensembl Variant Effect Predictor and RefSeq databases.

### (2) Determination of the List of Known Causative Genes for POI

POI pathogenic genes were retrieved by searching the PubMed and OMIM databases for articles published up to December 2021 using gene-related terms such as “gene,” “genetic,” “mutation,” or “variant” in combination with POI-related terms such as “ovarian insufficiency,” “ovarian failure,” “ovarian dysgenesis,” “ovarian aging,” “ovarian dysfunction,” “gonadal failure,” “gonadal dysgenesis,” “reproductive dysfunction,” or “gonadal insufficiency.” Genes were required to meet the following criteria: \textcircled{1} Such harmful mutations have been previously identified in POI patients; \textcircled{2} The relationship between the gene and the disease has been demonstrated through animal models or in vitro experiments, or there is strong genetic evidence, e.g., co-segregation in large POI families. Based on the requirements above, a total of 95 known POI pathogenic genes were screened, and the relevant phenotypic references for each known POI gene were also listed in the table below.

| Genes | References |
|---|---|
| AARS2 | PMID: 24808023 |
| ACAD9 | PMID: 26669660 |
| AIRE | PMID: 9398840; 23000069 |
| ALOX12B | PMID: 32253496 |
| AMH | PMID: 25750103 |
| AMHR2 | PMID: 12574214; 27430550 |
| ANKRD31^{a} | PMID: 34247419 |
| ATG7 | PMID: 30224786 |
| ATG9A | PMID: 30224786 |
| ATM | PMID: 7792600; 6050359 |
| BLM | PMID: 24602044; 29056561; 28232778 |
| BMP15 | PMID: 15136966 |
| BMPR1A | PMID: 31769494 |
| BMPR1B | PMID: 15805157; 31769494 |
| BNC1 | PMID: 30010909 |
| BRCA2 | PMID: 30207912; 30865812; 30865813; 32482800 |
| BUB1B | PMID: 32716490 |
| C14orf39 | PMID: 33508233 |
| CLPP | PMID: 23541340 |
| COX10 | PMID: 24100867 |
| CYP17A1 | PMID: 8855840; 34724156 |
| CYP19A1 | PMID: 8200927 |
| DIAPH2 | PMID: 9497258 |
| DMC1 | PMID: 29331980 |
| EIF2B2 | PMID: 12707859; 21484434 |
| EIF2B4 | PMID: 12707859 |
| EIF2B5 | PMID: 12707859; 21484434; 33245593 |
| EIF4ENIF1 | PMID: 23902945; 31810472 |
| ERCC6 | PMID: 26218421 |
| ESR2 | PMID: 30113650 |
| EXO1^{a} | PMID: 32772095 |
| FANCA | PMID: 24438373; 22294495; 24045675; 31535215 |
| FANCC | PMID: 24438373 |
| FANCG | PMID: 24438373; 32529760 |
| FANCL^{a} | PMID: 32048394 |
| FANCM | PMID: 29231814 |
| FIGLA | PMID: 18499083; 29914564 |
| FMR1 | PMID: 9647544 |
| FOXE1 | PMID: 16481406; 22177572 |
| FOXL2 | PMID: 11175783; 15459170; 21862621 |
| FSHR | PMID: 7553856; 26911863; 30691934 |
| GALT | PMID: 10573007; 17486650 |
| GDF9 | PMID: 33797006 |
| GNAS | PMID: 1944469; 20979189 |
| HARS2 | PMID: 21464306; 26970254 |
| HAX1 | PMID: 28681255; 23050867 |
| HFM1 | PMID: 24597873; 31279343 |
| HSD17B4 | PMID: 20673864; 28830375 |
| HSF2BP | PMID: 32845237 |
| KHDRBS1 | PMID: 28938739; 29808484 |
| LARS2 | PMID: 23541342 |
| LHCGR | PMID: 8559204; 30016538 |
| LMNA | PMID: 19283854 |
| LRPPRC | PMID: 28284481 |
| MCM8 | PMID: 25437880; 27573988; 27802094; 28863940; |
| | 32048466; 32652893 |
| MCM9 | PMID: 25480036; 26771056; 27802094; 32145932 |
| MEIOB | PMID: 31000419 |
| MRPS22 | PMID: 29566152 |
| MSH4 | PMID: 28541421 |
| MSH5 | PMID: 28175301 |
| NANOS3 | PMID: 24091668; 25054146 |
| NBN | PMID: 20444919; 29706645 |
| NOBOX | PMID: 17701902; 21837770; 25514101; 27603904; |
| | 27836978 |
| NOG | PMID: 15066478; 22088931 |
| NOTCH2 | PMID: 30304577; 32312275 |
| NR5A1 | PMID: 19246354; 23153500; 24073220; 25099250; |
| | 31787151 |
| NSMCE2 | PMID: 25105364 |
| NUP107 | PMID: 26485283 |
| PGRMC1 | PMID: 18782852 |
| PMM2 | PMID: 9140401; 25497157; 31902100 |
| POF1B | PMID: 16773570; 25676666 |
| POLG | PMID: 16595552, 29992832, 33538981 |
| POLR2C | PMID: 29367954 |
| POLR3H | PMID: 30830215 |
| POR | PMID: 14758361; 19837910; 32242900 |
| PRDM9^{a} | PMID: 34247419 |
| PSMC3IP | PMID: 21963259; 29240891 |
| RAD51^{a} | PMID: 32772095 |
| RCBTB1 | PMID: 27486781 |
| RECQL4 | PMID: 1122657; 1430398; 35086131 |
| SALL4 | PMID: 30603774 |
| SGO2 | PMID: 27629923 |
| SOHLH1 | PMID: 25774885; 27603904 |
| SPATA22^{a} | PMID: 35285020 |
| SPIDR | PMID: 27967308 |
| STAG3 | PMID: 24597867; 26059840; 28393351; 31803224; |
| | 32634216 |
| STAR | PMID: 9141542 |
| SYCE1 | PMID: 25062452 |
| SYCP2L | PMID: 32303603 |
| TP63 | PMID: 30924587; 32067224; 33452256 |
| TWNK | PMID: 25355836; 31455392; 31852434 |
| WDR62 | PMID: 30102701 |
| WRN | PMID: 20075015; 23849162 |
| WT1 | PMID: 26358501; 34845858 |
| XRCC2 | PMID: 30042186; 30489636 |

### (3) Determination of the Harmfulness of Mutations

The mutations of the above-noted genes identified in POI patients were classified by the ACMG ratings: Pathogenic (P), likely pathogenic (LP), and variant of uncertain significance (VUS). Mutations rated as VUS were verified by functional experiments.

Since BLM, HFM1, MCM8, MCM9, MSH4 and RECQL4 genes were all DNA homologous recombination repair genes, we used a homologous recombination (HR) reporter system to detect the HR efficiency of the mutations identified in BLM, HFM1, MCM8, MCM9, MSH4, and RECQL4, finding that certain mutations could lead to a decrease in the HR efficiency of the protein. Based on the ACMG rating, such mutation sites can be upgraded from VUS to LP. Given that the NR5A1 gene possesses transcriptional factor activity, we used the dual-luciferase reporter system to perform functional validation on the identified mutations and found that some mutations led to impaired transcriptional activity. All above-noted experiments were repeated three times independently. Error bars indicate s.e.m., and the numbers indicate P values: *P<0.05. ** The P value<0.01.*** The P-value is <0.001; n.s.—No statistical significance.

Based on the functional experiment result, a total of 108 patients carrying 119 P/LP mutation sites of 45 known POI pathogenic genes were found in 1,030 POI patients, accounting for 10.4% (107/1,030) of the total screened population. The site information is as follows:

| | | Gene | |
|---|---|---|---|
| Cases | Genes | ID | Mutation site |
| POI-121 | GDF9 | 2661 | NM_005260.5: c.612del |
| POI-133 | AARS2 | 57505 | NM_020745.4: c.1360G > T |
| POI-136 | BLM | 641 | NM_001287246.2: c.1949C > T |
| POI-173 | RECQL4 | 9401 | NM_004260.3: c.3034T > C |
| POI-175 | MCM8 | 84515 | NM_001281521.1: c.1553G > A |
| POI-184 | BMP15 | 9210 | NM_005448.2: c.373C > T |
| POI-188 | PMM2 | 5373 | NM_000303.3: c.526G > A |
| POI-201 | FIGLA | 344018 | NM_001004311.3: c.385-2A > G |
| POI-219 | SPIDR | 23514 | NM_001080394.4: c.2102del |
| POI-277 | RECQL4 | 9401 | NM_004260.3: c.2886-1G > A |
| POI-347 | HFM1 | 164045 | NM_001017975.5: c.3698C > T |
| POI-379 | WRN | 7486 | NM_000553.6: c.1270-1G > A |
| POI-402 | RECQL4 | 9401 | NM_004260.3: c.3515A > G |
| | MSH4 | 4438 | NM_002440.4: c.911dup |
| POI-444 | HFM1 | 164045 | NM_001017975.5: c.2336A > C |
| POI-465 | GDF9 | 2661 | NM_005260.5: c.333C > G |
| POI-492 | LMNA | 4000 | NM_170707.4: c.991C > T |
| POI-516 | SPIDR | 23514 | NM_001080394.4: c.2209C > T |
| | HFM1 | 164045 | NM_001017975.5: c.1792G > C |
| | HFM1 | 164045 | NM_001017975.5: c.3784G > A |
| POI-548 | FIGLA | 344018 | NM_001004311.3: c.385-2A > G |
| POI-561 | HSF2BP | 11077 | NM_007031.2: c.291 + 1G > T |
| POI-565 | MRPS22 | 56945 | NM_020191.4: c.1043_1046dup |
| POI-581 | HARS2 | 23438 | NM_001363535.2: c.567del |
| | BLM | 641 | NM_001287246.2: c.2980del |
| POI-584 | NR5A1 | 2516 | NM_004959.5: c.1262dup |
| POI-585 | SGO2 | 151246 | NM_152524.6: c.195dup |
| POI-631 | MCM8 | 84515 | NM_001281521.1: c.1857G > A |
| POI-658 | MCM9 | 254394 | NM_017696.2: c.398C > G |
| POI-695 | C14orf39 | 317761 | NM_174978.3: c.1037C > G |
| POI-700 | POF1B | 79983 | NM_001307940.2: c.1438-1G > T |
| POI-759 | BRCA2 | 675 | NM_000059.3: c.7084_7085del |
| POI-760 | C14orf39 | 317761 | NM_174978.3: c.1349dup |
| POI-808 | RECQL4 | 9401 | NM_004260.3: c.2128C > T |
| POI-811 | MCM9 | 254394 | NM_017696.2: c.78_80del |
| POI-835 | MCM9 | 254394 | NM_017696.2: c.1390G > A |
| POI-841 | MCM9 | 254394 | NM_017696.2: c.398C > G |
| | MCM9 | 254394 | NM_017696.2: c.1306A > G |
| POI-910 | RECQL4 | 9401 | NM_004260.3: c.2404G > T |
| | RECQL4 | 9401 | NM_004260.3: c.2554_2559dup |
| POI-916 | WDR62 | 284403 | NM_001083961.2: c.3203_3206del |
| POI-927 | HFM1 | 164045 | NM_001017975.5: c.3477A > C |
| POI-941 | WDR62 | 284403 | NM_001083961.2: c.178-2A > G |
| POI-942 | COX10 | 1352 | NM_001303.4: c.954_955del |
| POI-951 | HARS2 | 23438 | NM_001363535.2: c.1171_1183del |
| POI-956 | HFM1 | 164045 | NM_001017975.5: c.2028_2029del |
| POI-958 | AARS2 | 57505 | NM_020745.4: c.985del |
| POI-962 | NUP107 | 57122 | NM_020401.4: c.262C > T |
| POI-991 | AARS2 | 57505 | NM_020745.4: c.2005C > T |
| POI-992 | SPIDR | 23514 | NM_001080394.4: c.2656_2660del |
| POI-1012 | PSMC3IP | 29893 | NM_016556.4: c.597 + 1G > T |
| POI-1027 | FIGLA | 344018 | NM_001004311.3: c.1A > G |
| | NR5A1 | 2516 | NM_004959.5: c.821T > C |
| POI-1051 | BLM | 641 | NM_001287246.2: c.2310C > G |
| POI-1062 | HFM1 | 164045 | NM_001017975.5: c.1730A > G |
| POI-1106 | SPIDR | 23514 | NM_001080394.4: c.1174G > T |
| POI-1125 | AIRE | 326 | NM_000383.4: c.993del |
| POI-1130 | HSF2BP | 11077 | NM_007031.2: c.973G > T |
| POI-1134 | BMP15 | 9210 | NM_005448.2: c.542G > A |
| POI-1137 | EXO1 | 9156 | NM_130398.4: c.353_354del |
| POI-1146 | SPIDR | 23514 | NM_001080394.4: c.1181C > G |
| POI-1170 | HFM1 | 164045 | NM_001017975.5: c.3934C > T |
| POI-1171 | RECQL4 | 9401 | NM_004260.3: c.3418del |
| POI-1176 | BRCA2 | 675 | NM_000059.3: c.1910-1G > A |
| POI-1182 | RECQL4 | 9401 | NM_004260.3: c.1918C > G |
| POI-1211 | MCM8 | 84515 | NM_001281521.1: c.1111_1112del |
| | WDR62 | 284403 | NM_001083961.2: c.1255C > T |
| POI-1215 | GALT | 2592 | NM_000155.4: c.551del |
| POI-1228 | MCM9 | 254394 | NM_017696.2: c.1151-1G > A |
| | MCM9 | 254394 | NM_017696.2: c.322G > T |
| POI-1238 | RECQL4 | 9401 | NM_004260.3: c.2233C > T |
| POI-1247 | AARS2 | 57505 | NM_020745.4: c.298C > T |
| POI-1251 | MCM9 | 254394 | NM_017696.2: c.1390G > A |
| POI-1287 | POF1B | 79983 | NM_001307940.2: c.137dup |
| | MSH4 | 4438 | NM_002440.4: c.2546A > T |
| POI-1294 | CLPP | 8192 | NM_006012.4: c.6G > A |
| POI-1336 | STAR | 6770 | NM_000349.3: c.403G > T |
| POI-1339 | FANCM | 57697 | NM_020937.4: c.5653C > T |
| POI-1356 | GNAS | 2778 | NM_080425.3: c.344G > A |
| POI-1391 | FOXL2 | 668 | NM_023067.4: c.427dup |
| POI-1399 | TP63 | 8626 | NM_003722.5: c.1703del |
| POI-1402 | AIRE | 326 | NM_000383.4: c.1182C > A |
| POI-1406 | PMM2 | 5373 | NM_000303.3: c.492del |
| POI-1426 | RECQL4 | 9401 | NM_004260.3: c.2288G > A |
| POI-1431 | SPATA22 | 84690 | NM_001321337.1: c.400C > T |
| POI-1435 | POLG | 5428 | NM_001126131.2: c.1168del |
| POI-1451 | SPIDR | 23514 | NM_001080394.4: c.3G > A |
| POI-1453 | MSH4 | 4438 | NM_002440.4: c.1345T > G |
| | MSH4 | 4438 | NM_002440.4: c.1094T > A |
| POI-1474 | AIRE | 326 | NM_000383.4: c.44G > A |
| POI-1479 | FANCM | 57697 | NM_020937.4: c.1213C > T |
| POI-1480 | ATM | 472 | NM_000051.3: c.5830del |
| POI-1482 | BLM | 641 | NM_001287246.2: c.2693G > A |
| POI-1490 | BLM | 641 | NM_001287246.2: c.1019A > G |
| POI-1494 | HFM1 | 164045 | NM_001017975.5: c.1130A > G |
| POI-1503 | BNC1 | 646 | NM_001717.4: c.2476C > T |
| POI-1512 | PMM2 | 5373 | NM_000303.3: c.448-2A > G |
| POI-1520 | NR5A1 | 2516 | NM_004959.5: c.292A > T |
| POI-1583 | STAG3 | 10734 | NM_001282717.1: c.2938del |
| POI-1601 | HARS2 | 23438 | NM_001363535.2: c.1171_1183del |
| POI-1603 | MCM9 | 254394 | NM_017696.2: c.3160A > C |
| POI-1605 | HFM1 | 164045 | NM_001017975.5: c.1153A > G |
| POI-1655 | MSH4 | 4438 | NM_002440.4: c.1603C > T |
| POI-1707 | MCM8 | 84515 | NM_001281521.1: c.1385C > A |
| POI-1741 | MRPS22 | 56945 | NM_020191.4: c.1043_1046dup |
| POI-1753 | MCM9 | 254394 | NM_017696.2: c.816T > G |
| POI-1763 | NR5A1 | 2516 | NM_004959.5: c.245-2A > G |
| POI-1764 | FSHR | 2492 | NM_000145.4: c.447-1G > C |
| POI-1781 | HARS2 | 23438 | NM_001363535.2: c.567del |
| POI-20026 | CLPP | 8192 | NM_006012.4: c.368-3_368-2dup |
| POI-20032 | MSH4 | 4438 | NM_002440.4: c.2179G > C |
| | WRN | 7486 | NM_000553.6: c.−76-1G > A |
| POI-20058 | SYCP2L | 221711 | NM_001040274.3: c.1219-1G > C |
| POI-20227 | POLG | 5428 | NM_001126131.2: c.2677T > C |
| | STAR | 6770 | NM_000349.3: c.125del |
| POI-20290 | RECQL4 | 9401 | NM_004260.3: c.3062_3079dup |
| POI-20329 | FANCL | 55120 | NM_001114636.1: c.555 + 2T > G |
| POI-20437 | MCM9 | 254394 | NM_017696.2: c.595T > G |
| POI-20438 | ERCC6 | 2074 | NM_000124.4: c.1821 + 1G > T |
| POI-20548 | NUP107 | 57122 | NM_020401.4: c.8 + 1G > C |

(4) New POI-Associated Genes were Identified by Case-Control Analysis

Firstly, we screened 703 candidate genes associated with ovarian function from the genome, adhering to the following specific screening criteria: 1) The biological processes where genes are involved in follicle genesis and development; 2) According to the databases of Mouse Genome Informatics (MGI, http://www.informatics.jax.org/) and International Mouse Phenotype Consortium (https://www.mousephenotype.org/), deficiency of this gene may lead to impaired ovarian function in mice; 3) In vitro studies or other animal models have demonstrated that this gene is functionally related to ovarian function-related pathways.

For the 703 genes screened, we performed burden analysis in 1030 POI patients and 5000 controls using two models, i.e., LoF and D-mis models. The LoF model only included LoF variants (start-loss, canonical splice-site, frame shift, and nonsense). For the D-mis model, a variety of algorithms were used to predict the harmfulness of missense variants, and six criteria were set up to define D-mis: \textcircled{1}With SIFT<0.05, Polyphen2>0.15, and MutationTaster predicted as harmful; \textcircled{2}Predicted as “possibly pathogenic” by M-CAP; \textcircled{3}Predicted as “harmful” by MetaSVM; \textcircled{4}REVEL>0.75; \textcircled{5}CADD>20; and \textcircled{6}CADD>10. Parallel analyses were performed on the D-mis determined by different criteria, and their results were compared. The number of alleles in cases and controls was compared using unilateral Fisher's exact test. Significantly mutation-enriched genes were identified in POI patients according to “P<0.05.”

### (5) Sanger Sequencing to Verify Gene Variants

The suspected pathogenic mutations of the candidate genes were identified through above-noted screening, and the mutation accuracy was verified by Sanger sequencing.

The newly discovered POI pathogenic genes and mutation sites are as follows:

| | | Gene | |
|---|---|---|---|
| Cases | Genes | ID | Mutation site |
| POI-149 | PPM1B | 5495 | NM_002706.6: c.1253dup |
| POI-169 | ALOX12 | 239 | NM_000697.3: c.1909G > T |
| POI-270 | LGR4 | 55366 | NM_018490.5: c.2449C > T |
| POI-362 | MCMDC2 | 157777 | NM_173518.5: c.204del |
| POI-374 | ALOX12 | 239 | NM_000697.3: c.542 + 1G > A |
| POI-405 | HSD17B1 | 3292 | NM_001330219.3: c.313G > T |
| POI-410 | BMP6 | 654 | NM_001718.6: c.472C > T |
| POI-439 | SHOC1 | 158401 | NM_173521.5: c.3903G > A |
| POI-446 | CCDC155 | 147872 | NM_144688.5: c.1269 + 2T > A |
| POI-450 | SHOC1 | 158401 | NM_173521.5: c.231_232del |
| POI-480 | CCDC155 | 147872 | NM_144688.5: c.218del |
| POI-482 | HMMR | 3161 | NM_001142556.2: c.1386-2A > C |
| POI-502 | RFWD3 | 55159 | NM_001370535.1: c.789del |
| POI-539 | STRA8 | 346673 | NM_182489.1: c.258 + 1G > A |
| POI-567 | LGR4 | 55366 | NM_018490.5: c.903-3_903-2del |
| POI-677 | PRDM1 | 639 | NM_001198.4: c.1866C > G |
| POI-710 | MST1R | 4486 | NM_002447.4: c.1480_1481del |
| POI-785 | ALOX12 | 239 | NM_000697.3: c.805C > T |
| POI-791 | HSD17B1 | 3292 | NM_001330219.3: c.721-1G > C |
| POI-915 | HSD17B1 | 3292 | NM_001330219.3: c.947dup |
| POI-962 | MST1R | 4486 | NM_002447.4: c.3846del |
| POI-966 | NUP43 | 348995 | NM_198887.3: c.641del |
| POI-1012 | ZAR1 | 326340 | NM_175619.3: c.1131 + 2T > C |
| POI-1096 | SLX4 | 84464 | NM_032444.4: c.100C > T |
| POI-1112 | RFWD3 | 55159 | NM_001370535.1: c.1561_1562del |
| POI-1170 | SHOC1 | 158401 | NM_173521.5: c.1939 + 2T > A |
| POI-1169 | HMMR | 3161 | NM_001142556.2: c.667G > T |
| POI-1182 | ZP3 | 7784 | NM_001110354.2: c.52G > T |
| POI-1191 | HSD17B1 | 3292 | NM_001330219.3: c.313G > T |
| POI-1251 | ZP3 | 7784 | NM_001110354.2: c.1028G > A |
| POI-1356 | CPEB1 | 64506 | NM_001365240.1: c.1438_1444del |
| POI-1399 | ZP3 | 7784 | NM_001110354.2: c.633_652del |
| POI-1406 | SHOC1 | 158401 | NM_173521.5: c.3101_3102dup |
| POI-1426 | SLX4 | 84464 | NM_032444.4: c.4390_4393dup |
| POI-1346 | ZAR1 | 326340 | NM_175619.3: c.119G > A |
| POI-1463 | MEIOSIN | 388553 | NM_001310124.2: c.654del |
| POI-1481 | HSD17B1 | 3292 | NM_001330219.3: c.97 + 1G > A |
| POI-1496 | CPEB1 | 64506 | NM_001365240.1: c.1140_1144 + |
| | | | 10del |
| POI-1528 | ZAR1 | 326340 | NM_175619.3: c.270_297dup |
| POI-1544 | ZAR1 | 326340 | NM_175619.3: c.44dup |
| POI-1551 | H1FOO | 132243 | NM_153833.2: c.251_261del |
| POI-1561 | SLX4 | 84464 | NM_032444.4: c.928C > T |
| POI-1601 | ZAR1 | 326340 | NM_175619.3: c.1132-2A > C |
| POI-1590 | H1FOO | 132243 | NM_153833.2: c.212del |
| POI-1626 | NUP43 | 348995 | NM_198887.3: c.544C > T |
| POI-1640 | MCMDC2 | 157777 | NM_173518.5: c.685C > T |
| POI-1660 | ZAR1 | 326340 | NM_175619.3: c.270_297dup |
| | ZAR1 | 326340 | NM_175619.3: c.1108C > T |
| POI-1681 | HMMR | 3161 | NM_001142556.2: c.145 + 1G > A |
| POI-1689 | MST1R | 4486 | NM_002447.4: c.3991C > T |
| POI-1694 | CCDC155 | 147872 | NM_144688.5: c.876 + 1G > A |
| POI-1705 | PPM1B | 5495 | NM_002706.6: c.644_671del |
| POI-1741 | PRDM1 | 639 | NM_001198.4: c.2322_2325dup |
| POI-1717 | SHOC1 | 158401 | NM_173521.5: c.231_232del |
| POI-1734 | BMP6 | 654 | NM_001718.6: c.1141C > T |
| POI-1775 | HMMR | 3161 | NM_001142556.2: c.429del |
| POI-1787 | MEIOSIN | 388553 | NM_001310124.2: c.214A > T |
| POI-20272 | LGR4 | 55366 | NM_018490.5: c.1580-2dup |
| POI-20357 | PRDM1 | 639 | NM_001198.4: c.32del |
| POI-20363 | MST1R | 4486 | NM_002447.4: c.1230 + 2T > C |
| POI-20533 | SHOC1 | 158401 | NM_173521.5: c.143_146del |
| POI-20556 | NUP43 | 348995 | NM_198887.3: c.639del |

### 4. Verification of the Harmful Function of New POI Associated Gene Mutations:

### (1) Verification of Pathogenicity of PRDM1 Gene Mutations

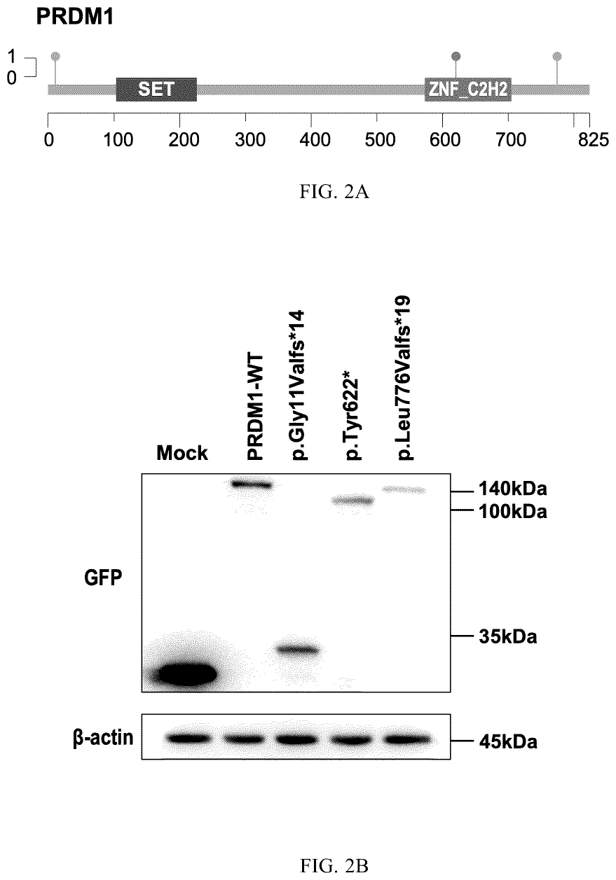

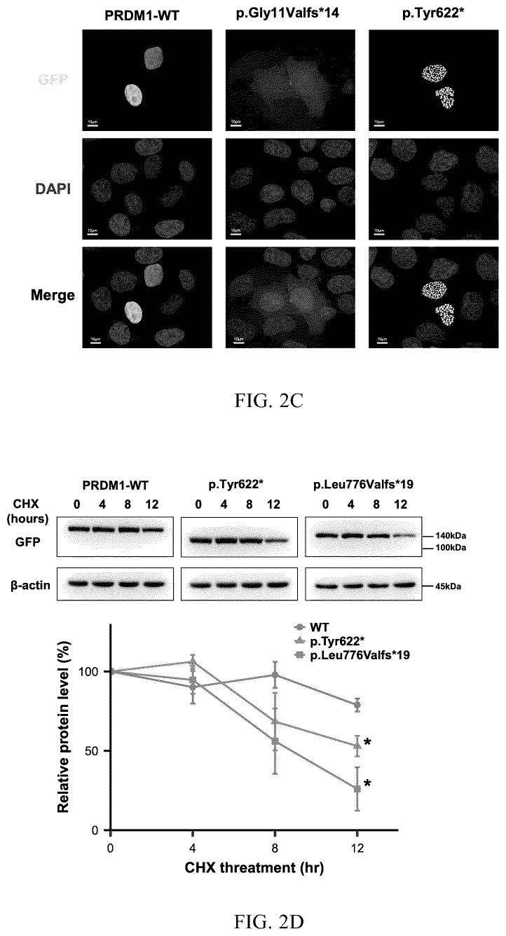

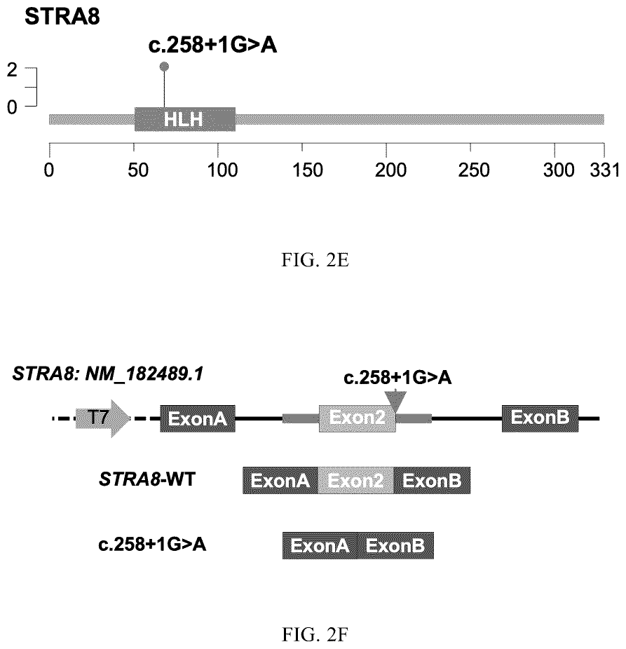

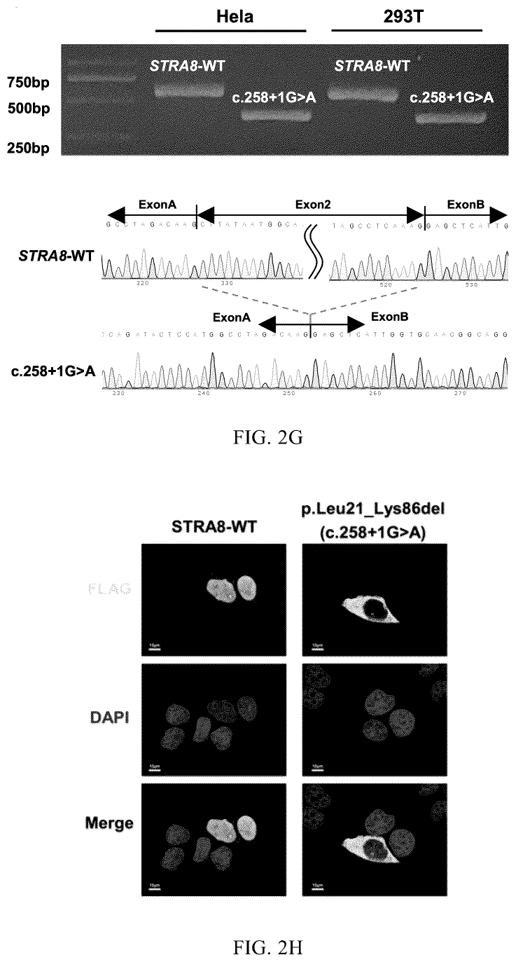

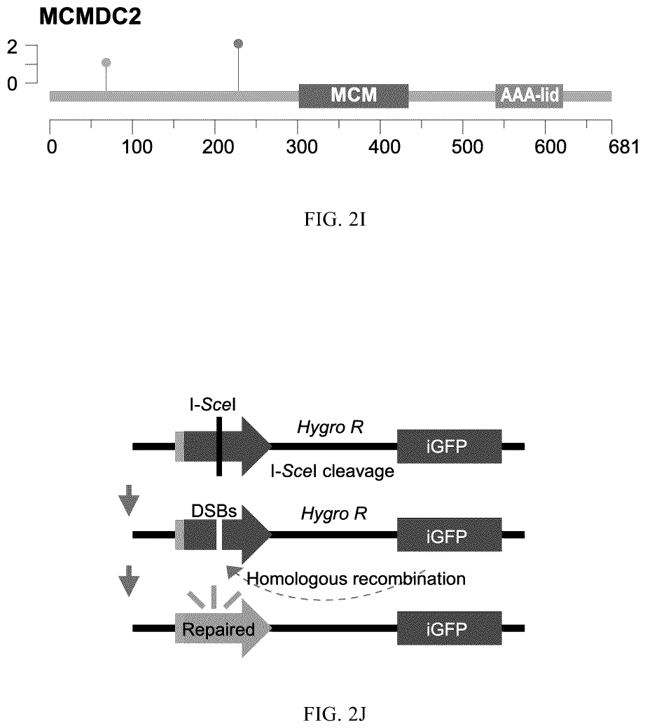

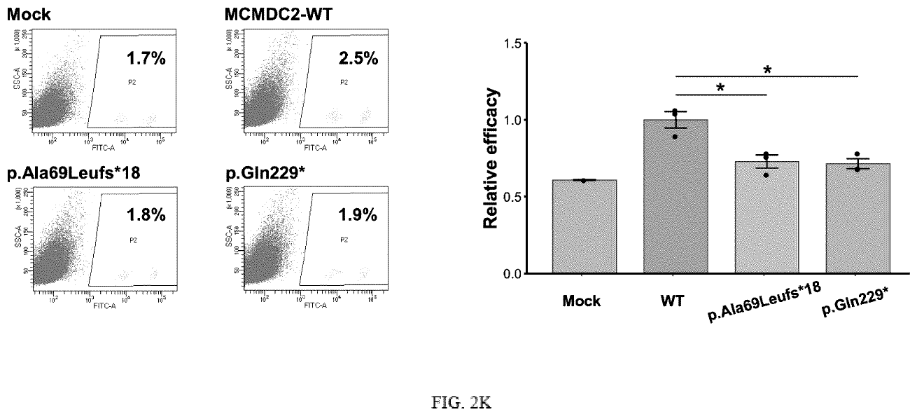

PRDM1 is a key transcriptional regulator required for proliferation and migration of primitive germ cells. In this embodiment, three PRDM1 heterozygous mutations were detected, including p.GlyllValfs*14, p.Tyr622*, and p. Leu776Valfs*19, all of which were lost-of-function mutations. Secondary amenorrhea occurred in all three mutation carriers, and B-ultrasonography indicated bilateral ovarian atrophy with almost no residual follicles. We then performed functional experiments to verify the pathogenicity of these three PRDM1 mutations (FIG. 2A). Western blot experiments suggested that mutations PLY11valFS *14 and p.Tyr622* resulted in protein truncation, while P.LY776valFS *19 further reduced protein expression (FIG. 2B). Meanwhile, cell overexpression experiments showed that, unlike the uniform nuclear distribution of the wild-type PRDM1-GFP fusion protein, the mutant protein p. Gly11Valfs*14 was expressed in both nucleus and cytoplasm, resembling the pEGFP empty vector group; in contrast, mutant protein p.Tyr622* aggregated in a punctate pattern, suggesting that the mutant protein may lose its ability to bind to DNA, resulting in exceptional self-aggregation of the protein (FIG. 2C). Furthermore, ethylene oxide experiments showed that after inhibition of protein synthesis, the degradation rate of mutant protein p.Leu776Valfs*19 significantly increased, suggesting that the mutation resulted in a decrease in the stability of PRDM1 protein (FIG. 2D). The experiments above confirmed that the mutation affected the function of PRDM1 protein, which may lead to a decrease in ovarian reserve and cause POI by affecting the proliferation and migration of primitive germ cells.

### (2) Verification of Pathogenicity of STRA8 Gene Mutations

STRA8 is a gene that is specifically highly expressed in the early stage of meiosis in germ cells. It exerts transcriptional regulatory functions and promotes the expression of meiosis-related genes, thereby triggering the initiation of meiosis. In this embodiment, 1 splicing site mutation c.258+1G>A close to exon 2 was found in 1 patient with primary amenorrhea (FIG. 2E), while no loss-of-function variant of biallelic was found in 5000 cases. In addition, no biallelic loss-of-function mutations of this gene were found in any public databases, such as gnomAD, ExAC, and 1000 Genomes. The rare variant carried by this POI patient suggested that the STRA8 gene was closely associated with POI. Subsequently, we confirmed by Minigene experiments that mutation c.258+1G>A caused exon 2 skipping in the STRA8 gene, resulting in a 66-amino acid in-frame deletion (p.Leu21_Lys86del) in the most highly conserved region of STRA8, which is very important for STRA8 nuclear localization and binding to DNA (FIGS. 2F and 2G). Further immunofluorescence staining indicated that P. Leu21_Lys86del mutated STRA8 was confined to the cytoplasm (FIG. 2H), which was consistent with protein localization in mouse models of Stra8 N-terminal deletion. The cytoplasmic localization of STRA8 suggests that its transcriptional activation has been lost, thus hindering the initiation of meiosis. In view of the phenotype of ovarian atrophy and excessive follicle loss in Stra8 knockout mice, our findings in POI patients suggest that meiosis abnormalities caused by STRA8 gene mutation can be used as an indicator for POI etiology screening.

### (3) Verification of Pathogenicity of MCMDC2 Gene Mutations

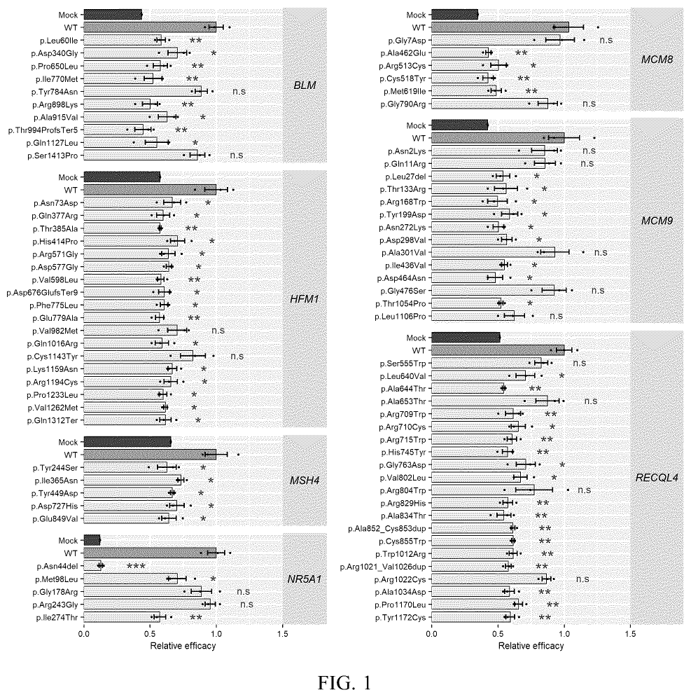

In the prophase of meiosis, MCMDC2 promotes homologous chromosome pairing and the formation of recombination complexes. promotes homologous chromosome pairing and the formation of recombination complexes. We found that 2 patients with secondary amenorrhea carried p.G 229* mutation and p.A 69LEUFS *18 mutation, both of which resulted in the loss of MCMDC2 key functional domains, MCM and AAA-lid (FIG. 1). Further green fluorescent protein (GFP)-based homologous recombination (HR) repair efficiency assays revealed that both mutant proteins had only 20% of the HR efficiency of the wild-type protein in reporter HEK293 cells (FIGS. 2J-K). This suggests that both mutants could hinder the progression of meiostic homologous recombination, which in turn led to abnormal oocyte meiosis and oocyte apoptosis.

### (4) Verification of Pathogenicity of SHOC1 Gene Mutations

During meiotic homologous recombination, SHOC1 is involved in stabilizing recombination intermediates and promoting the formation of crossover and synaptonemal complexes. We identified a homozygous frame shift mutation p.Leu78Serfs*10 in a patient with POI. The patient began menarche at the age of 17 and soon developed oligomenorrhea and amenorrhea. In addition, we identified 4 heterozygous loss-of-function mutations: p. Gln48Leufs*3, p.Leu1035Glyfs*28, p.Trp1301*, and c.1939+2T>A. The mutations could lead to abnormal binding of SHOC1 to DNA by disrupting the conserved XPF endonuclease-like central structural domain and the C-terminal helix-hairpin-helix (HhH2) domain, thus hindering the process of meiotic homologous recombination and inducing oocyte apoptosis.

### (5) Heterozygous Loss-of-Function Mutations in Eight Meiosis Genes

MEIOSIN is another transcription factor that, together with STRA8, drives the initiation of meiosis. Two variants, p.Thr221ProfsTer32 and p.Lys72Ter, were identified in MEIOSIN, which disrupted the HLH and HMG DNA-binding domains, respectively, thereby affecting the transcriptional activation of meiosis genes.

CCDC155 is involved in rapid chromosome movement and homologous pairing during meiosis. The three mutations identified in the present study, c.876+1G>A, c.1269+2T>A, and p.Arg73ProfsTer42, may lead to abnormal binding of CCDC155 to dynein through the loss of large C-terminal protein fragments, resulting in exceptional chromosomal motility.

CPEB1 promotes the translation of SYCP1/3 genes and the progression of meiosis. Large fragment deletion of CPEB1 has previously been reported in patients with POI, but the evidence for relevance was inconclusive. The present study identified two short deletions, c.1140_1144+10del and c.1438_1444del (p.Val480Profs*17). Prediction results suggested that the mutations could lead to abnormal RNA-protein interactions and protein recruitment during ribonucleoprotein complex assembly, resulting in unstable mRNA levels of genes that regulate meiosis and folliculogenesis.

NUP43 is a component of the coding nuclear complex, and its subunit, NUP107, has been reported to cause ovarian dysplasia through defective meiotic DNA damage response. The three mutations in NUP43, i.e., p.Thr214MetfsTer20, p.Arg182Ter and p.Thr214LeufsTer20, all located in the conserved WD40 structural domain, are predicted to compromise stability of the nucleoprotein complex and affect ovarian reserve through meiotic abnormalities.

RFWD3 (FANCW) and SLX4 (FANCP) belong to the Fanconi anemia family of genes and are involved in meiosis. The homologous recombination repair process relies on the clearance of RPA and RAD51 from DNA damage sites, while two variants of RFWD3, p.Thr521TyrfsTer2 and p.Lys263AsnfsTer85, may reduce their interaction with RPA and RAD51. SLX4 is involved in the unwinding of Holliday junctions, a homologous recombination process, while mutations p.Gln34Ter, p.Arg1465GlnfsTer3, and p.Arg310Ter can cause aberrant dissolution of Holliday junctions by affecting the action of SLX4 on other nuclease enzymes and impede meiotic progression.

### (6) Loss-of-Function Mutations Found in Follicle Development-Related Genes

HMMR proteins interact with the spindle to affect granulosa cell division and follicular development. Four HMMR loss-of-function mutations were identified in this study: c.1386-2A>C, p.Glu223Ter, c.145+1G>A, and p.Glu143AspfsTer2, which can affect granulosa cell function and inhibit follicular development by hindering the binding of HMMR proteins to centrosomes.

HSD17B1 is a key ovarian steroidogenic enzyme that maintains estrogen production and granulosa cell function. In the present study, five patients were found to harbor four loss-of-function mutations in HSD17B1: c.97+1G>A, c.721-1G>C, p.Glu105Ter and p.Asp318GlyfsTer3. The aforementioned mutations can affect the dehydrogenase activity of short-chain dehydrogenase/reductase by disrupting its structural domain, resulting in abnormal hormone synthesis and abnormal granulosa cell function.

BMP6 is an ovary-specific ligand that affects follicular development through multiple mechanisms, including promoting the expression of AMH and FSHR. 2 patients in the present study were found to harbor 2 loss-of-function mutations: p.Gln158Ter and p.Arg381Ter. Both mutants resulted in loss of BMP6 activity due to truncation of the 2 proteins resulting in loss of the C-terminal ligand structure that regulates TGF-β signaling.

H1-8 is an oocyte-specific linker histone involved in promoting oocyte meiosis, maturation, and fertilization. The loss-of-function mutations identified in the study in patients with POI, i.e., p.Lys84SerfsTer64 and p.Gly71AlafsTer27 may lead to chromatin coagulation dysfunction by disrupting the histone junction domain of the H1-8 protein.

Mutations in the PPM1B and ALOX12 genes are associated with age at menarche or natural menopause. Depletion of PPM1B leads to premature cellular senescence through sustained activation of p53 and results in reduced ovulation in mice. Two loss-of-function mutations identified in our POI patients, p.Tyr418Ter and p.Pro215LeufsTer7, may inhibit the dephosphorylation of PPM1B. ALOX12 is a key enzyme in fatty acid lipogenesis metabolism and plays a role in ovulation. Three mutations of ALOX12, i.e., c.542+1G>A, p.Glu637Ter, and p.Gln269Ter, can disrupt its lipoxygenase domain, thereby affecting its enzyme activity.

MST1R is a surface receptor that affects ovulation by regulating nitrogen oxygen levels and inflammatory responses. A total of seven mutations were detected, including three variants found within the Sema domain required for ligand binding, three mutations that affect receptor activation by inhibiting the tyrosine kinase domain, and a frame shift mutation that loses both Sema and tyrosine kinase domains.

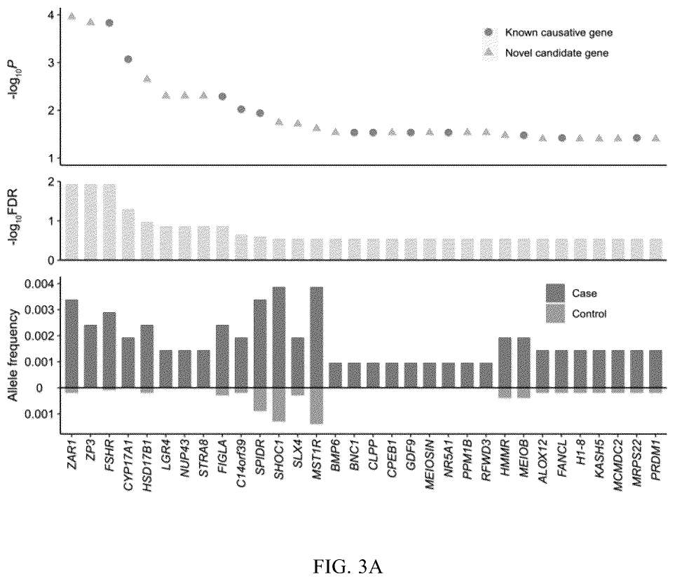

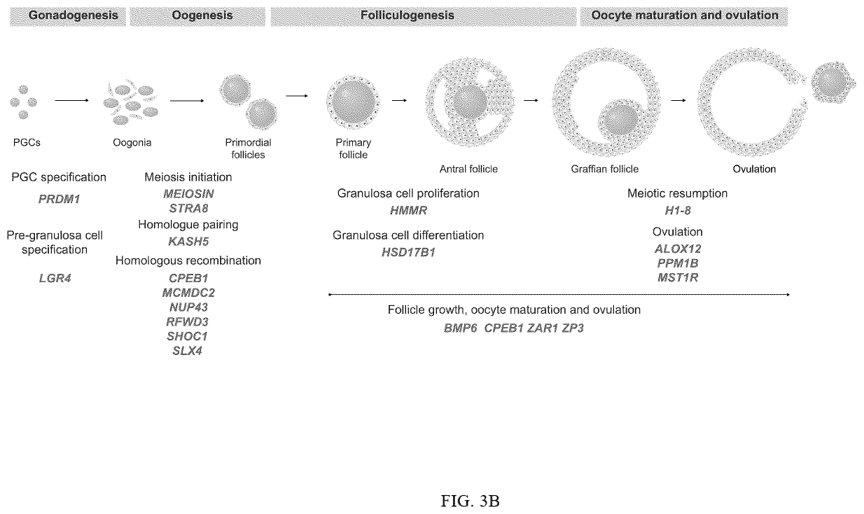

Functional annotations of above-noted 20 genes suggest a considerable correlation with POI, and there is a significant enrichment of loss-of-function mutations in POI patients, which can alter their protein levels or biological functions. This is exemplified by the experimental validation of PRDM1, STRA8 and MCMDC2 (FIGS. 3A and 3B). These data are strongly suggesting that these 20 new genes may be previously unrecognized POI pathogenic genes.

### Embodiment 2

In this embodiment, 9 heterozygous mutations in the TP63 gene were found in 11 patients by whole exome sequencing analysis of 1030 patients with idiopathic POI, and their pathogenicity was verified by functional experiments.

### 1. Subject

1030 patients with idiopathic POI were collected and their peripheral blood was sampled. The study was approved by the Ethics Committee of the Hospital for Reproductive Medicine Affiliated to Shandong University, and all study subjects signed the informed consent form. The inclusion and exclusion criteria for said patients with idiopathic POI are as follows:

Inclusion criteria: Primary or secondary amenorrhea before the age of 40 years; FSH levels>25 IU/L on two tests performed at an interval of more than 4 weeks.

Exclusion criteria: Patients with known causes of POI such as chromosomal abnormalities, history of ovarian surgery, history of radiotherapy and chemotherapy, and autoimmune diseases.

### 2. DNA Extraction

Genomic DNA from all samples was extracted from peripheral blood using the DNeasy Blood & Tissue Kit (Qiagen). The DNA was quantified using OD measurements (NanoDrop, Thermo Scientific) and 1 μg was taken for DNA library preparation.

### 3. Whole Exome Sequencing and Data Analysis

1 μg of genomic DNA was taken and DNA libraries were prepared using AIExome Enrichment Kit V1 (iGeneTech, Beijing, China). Exon capture was performed using SureSelect Target Enrichment System. The captured exome sequences were sequenced using the NovaSeq platform (Illumina HiSeq). Reads were subsequently aligned to the human genome reference sequence GRCh37/hg19 using Burrows-Wheeler Aligner MEM software. Duplicate Reads were removed and screened for SNPs and SNVs using Genome Analysis Toolkit (GATK) software. The variants screened were annotated using the Ensembl Variant Effect Predictor and RefSeq databases.

### 4. Interpretation of Variation Pathogenicity

Nine suspected pathogenic heterozygous mutations in the TP63 gene were identified in 11 patients with POI following the screening above. The variants identified were analyzed and classified with reference to the American College of Medical Genetics and Genomics Guideline (ACMG).

The amino acids at the 9 TP63 mutation sites were highly conserved across multiple species, and most of the mutations were evaluated as potentially pathogenic variants, as analyzed in Table 1.

TABLE 1
Function of TP63 Gene Mutation in 11 POI Patients

| | | | Amino | | gnomAD/ExAC | Conser- | | Mutation | Poly | | |
|---|---|---|---|---|---|---|---|---|---|---|---|
| No. | Exon | Variation | acid | Genotype | frequencyA | vatism | SIFT | Taster | Phen-2 | Cadd | ACMG^{B} |
| 1 | 6 | c.854G > A | p.S285N | Heterozygosis | 0.000006572/0.00001648 | Yes | D | D | PD | 4.534751 | VUS |
| 2 | 12 | c.1612A > G | p.T538A | Heterozygosis | 0.00002640/0.00003297 | Yes | D | D | D | 4.022315 | VUS |
| 3 | 13 | c.1700C > T | p.T567I | Heterozygosis | —/— | Yes | D | D | PD | 6.453378 | VUS |
| 4 | 13 | c.1703delA | p.Q568fs*3 | Heterozygosis | —/— | Yes | — | — | — | — | P |
| 5 | 14 | c.1780C > T | p.R594* | Heterozygosis | 0.00003290/0.00007419 | Yes | — | D | — | 12.739385 | P |
| 6 | 14 | c.1928G > A | p.R643Q | Heterozygosis | —/— | Yes | D | D | D | 6.809899 | P |
| 7 | 14 | c.1937T > C | p.L646P | Heterozygosis | 0.000006576/— | Yes | D | D | D | 5.886101 | P |
| 8, 9, | 14 | c.1939C > T | p.R647C | Heterozygosis | 0.00001316/— | Yes | D | D | D | 7.791268 | P |
| 10 | | | | | | | | | | | |
| 11 | 14 | c.1964G > A | p.R655Q | Heterozygosis | 0.00001315/0.00001649 | Yes | D | D | D | 7.516066 | P |

Reference sequences:
NM_003722.5; ACMG (American College of Medical Genetics and Genomics Guidelines; D, pathogenic; P, pathogenic variant; PD, possibly pathogenic; POI, premature ovarian insufficiency; VUS, variant of undetermined significance; “—”, not available or not applicable.
Afrequencies in gnomAD or ExAC database; ^{B}assessment of mutation pathogenicity after functional experiments.

### 5. Sanger Sequencing to Verify Gene Variants

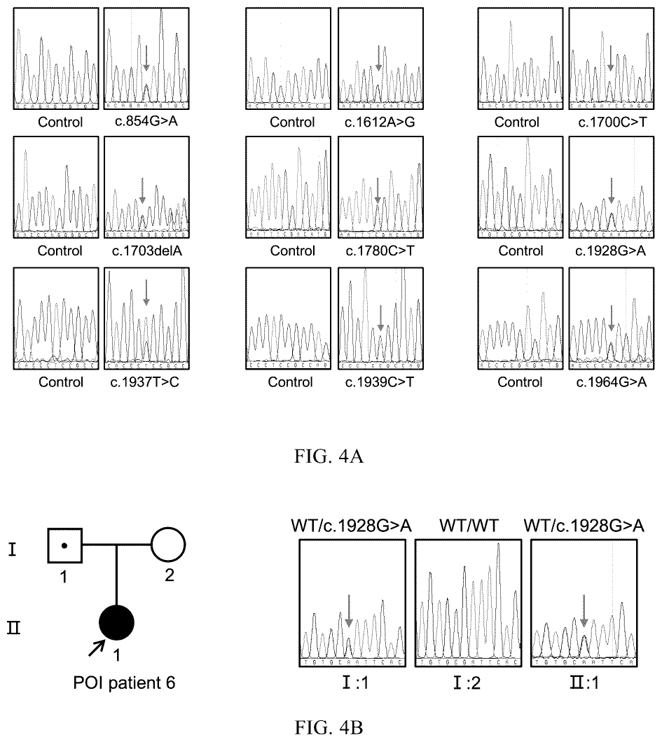

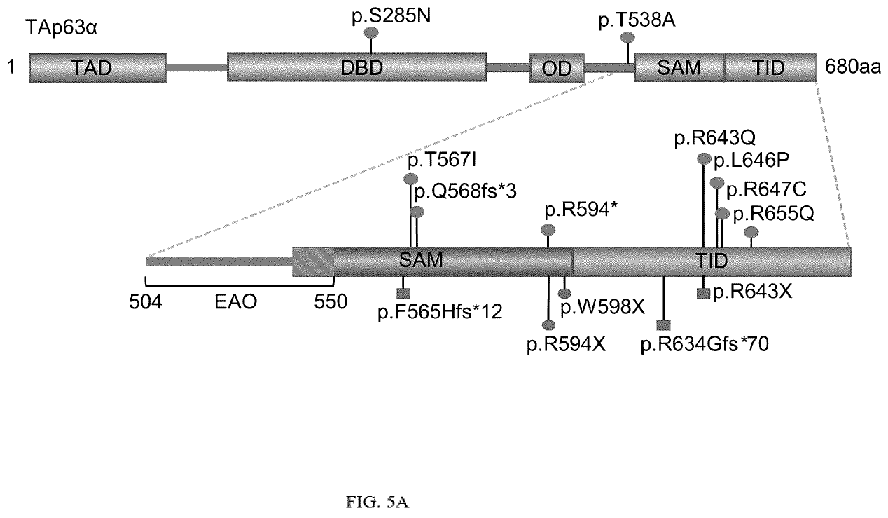

9 TP63 gene mutations were verified for accuracy by Sanger sequencing (FIG. 4A). Sanger sequencing of family members of POI patient No. 6 revealed that the mutation c.1964G>A was derived from her father (FIG. 4B).

### 6. In Vitro Functional Validation of Mutant Pathogenicity

### (1) Pathogenicity Analysis of Human TAp63α Mutations

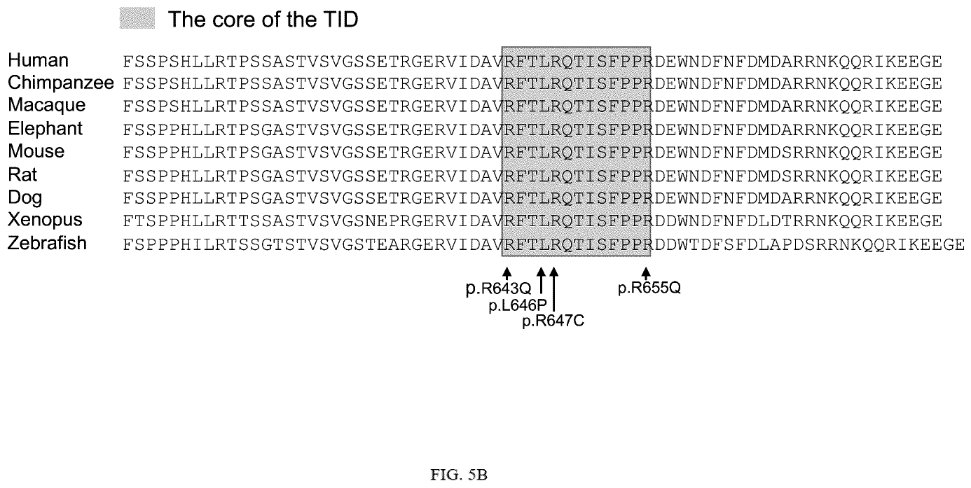

The TAp63α mutation sites detected in this embodiment were analyzed; as shown in FIG. 5A, the p.S285N and p.T567I mutations were located in the DNA binding domain (DBD) and SAM (sterile alpha motif) domains, respectively. The p.T538A mutation was not in any known functional domain, and the remaining 6 mutations all affected the C-terminal TID, a domain that is critical for the formation of inactivated dimers. Frame shift mutation p. Q568fs*3 and nonsense mutation p. R594* were located within the SAM domain, leading to loss of TID. Furthermore, 4 point mutations (P. R643Q, p.L646P, p.R647C, and p.R655Q) were located in the core sequence of TID from R643 to R655 (RFTLRQTISFPPR), which was responsible for binding to TAD (FIG. 5B). The analysis above suggests that mutations that impair TID may be cause of POI.

### (2) TAp63α Mutations Disrupt Protein Homeostasis

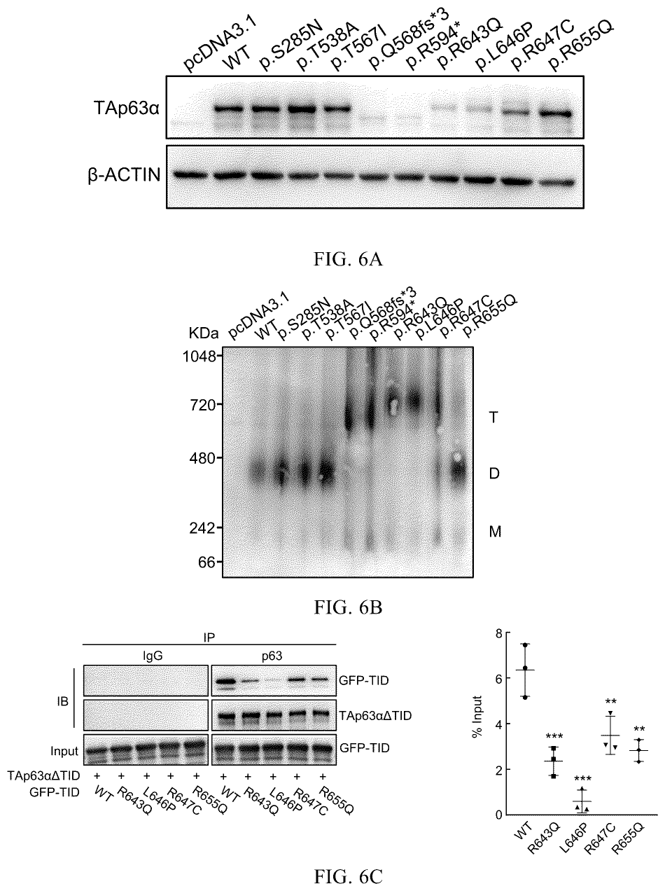

To further test the pathogenicity of mutant proteins, we overexpressed the wild-type (WT) and mutant TAp63α proteins in SAOS-2 cells (human osteosarcoma cells). Western blotting showed that WT and four mutant proteins (p.S285N, p.T538A, p.T567I, and p.R655Q) were highly expressed, while other mutant proteins were almost undetectable (p.Q568fs*3 and p.R655Q). R594*) or significantly reduced in expression (p.R643Q, p.L646P, and p.R647C) (FIG. 6A). Considering that the active form of TAp63α is difficult to detect due to its high turnover, these results suggest that some of above-noted mutant proteins may be spontaneously activated. Following this, to further investigate the polymeric status of these mutant proteins, we inhibited their proteasome-dependent degradation through MG132 treatment and performed BN-PAGE analysis. The results showed that all TID-related mutants (p.Q568fs3, p.R594, p.R643Q, p.L646P, p.R647C, and p.R655Q) led to tetramers, while other mutations (P. S285N, p.T538A and p.T567I) remain dimeric (FIG. 6B). This suggests that only the mutant proteins that affect TID were spontaneously activated. It is speculated based on previous studies that that four point mutations located in the core sequence of TID may disrupt the binding of TAD to TID, thereby altering the dimer conformation of TAp63α protein. To verify this hypothesis, we co-transfected TAp63αΔTID with plasmids of WT or mutant TID and performed co-immunoprecipitation experiments. The results suggested that compared with WT TID, the p.R643Q, p.L646P, p.R647C, and p.R655Q mutations of TID significantly reduced their interaction with TAp63α ΔTID (FIG. 6C). These results suggest that TID-related mutations disrupted the inactive dimer conformation of TAp63α and form activated tetramers.

### (3) TAp63α Mutant Protein Activates Apoptosis

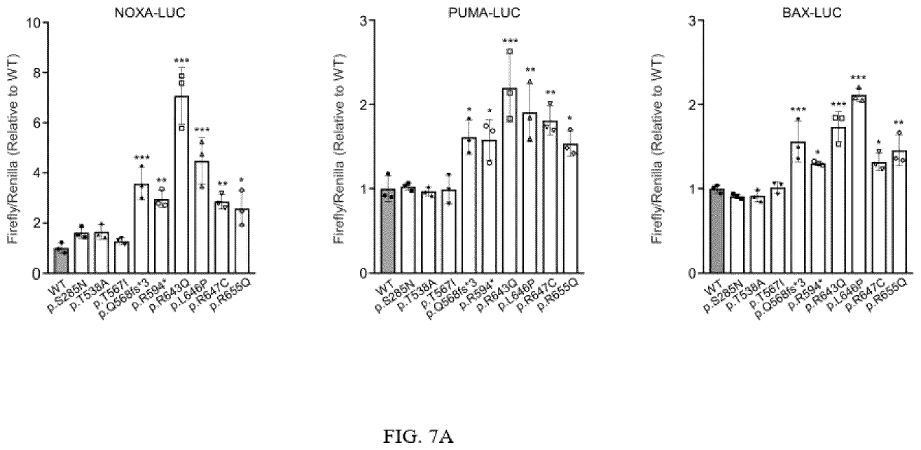

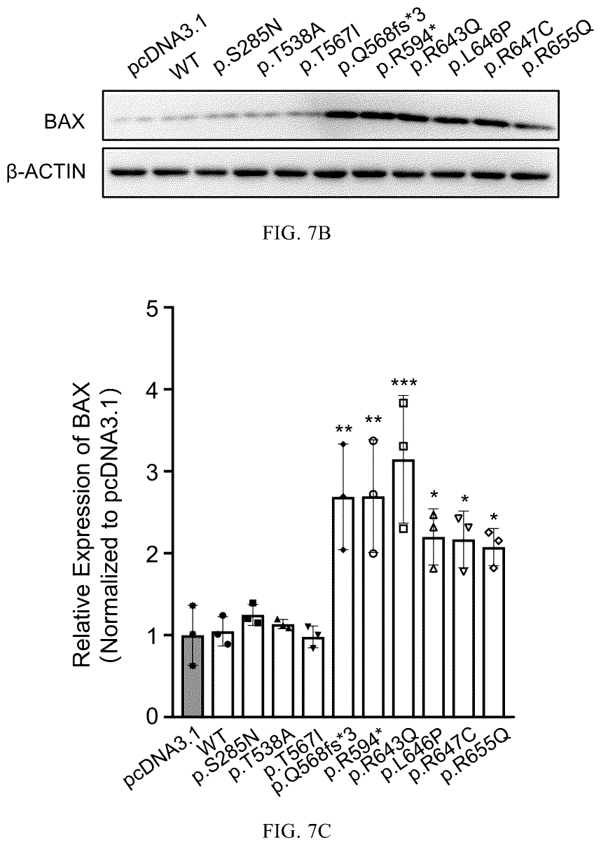

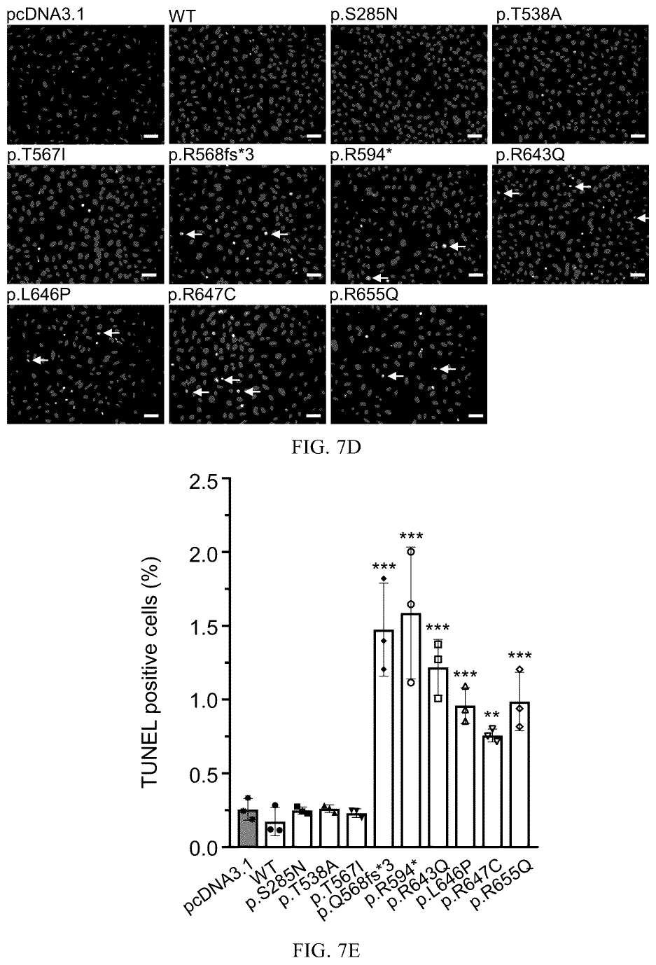

We evaluated the transcriptional activity of TAp63α by luciferase reporter gene assay. Compared with WT Tap63α, all six TID-related mutants (p.Q568fs3, p.R594, p.R643Q, p.L646P, p.R647C, and p.R655Q) exhibited transcriptional activation effects on the three gene promoter reporter plasmids, i.e., NOXA, PUMA, and BAX (FIG. 7A). Furthermore, six TID-associated mutants resulted in a significant increase in BAX protein expression (FIGS. 7B and 7C). Meanwhile, the proportion of TUNEL-positive cells increased significantly in SAOS-2 cells overexpressing TID-associated mutants (FIGS. 7D and 7E). The results above suggest that the mutations affecting TID spontaneously activate Tap63α, promoting the expression of its target genes and inducing apoptosis, ultimately leading to premature exhaustion of oocytes and POI.

### 7. In Vivo Functional Verification of Mutational Pathogenicity

(1) Building p63^{+/ΔTID} Mouse Model

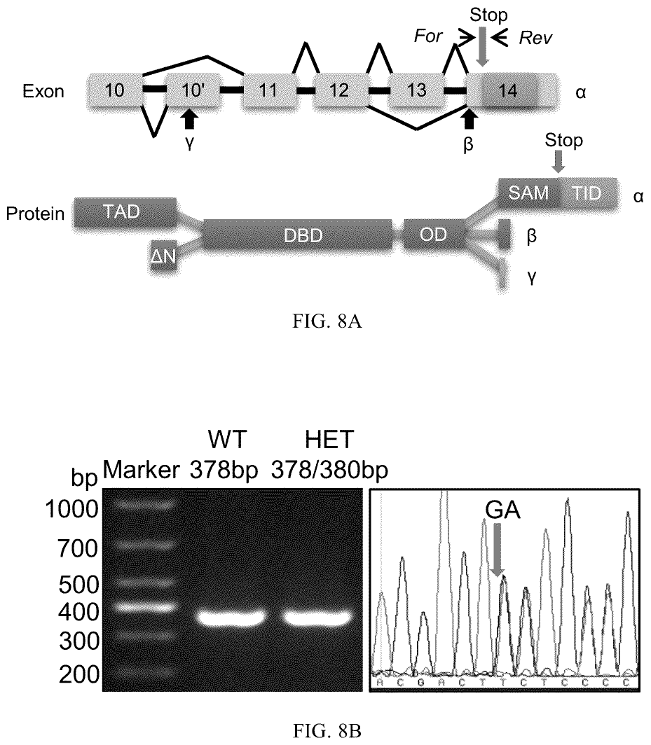

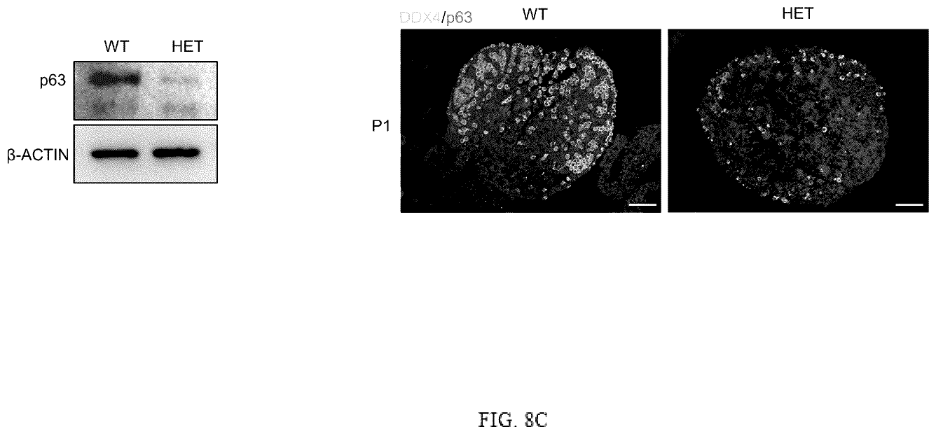

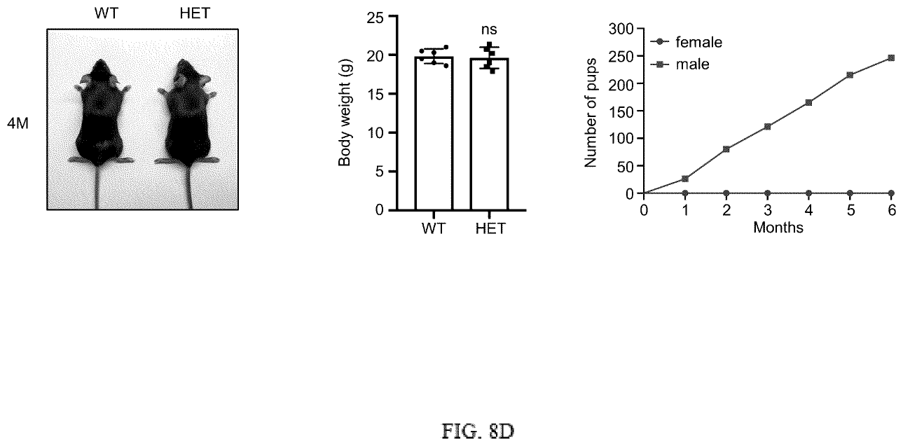

To determine the role of p63 TID in the pathogenesis of POI in vivo, we inserted two nucleotides in front of the TID of exon 14 of mouse p63 gene to introduce a stop codon, specifically knocking out the TID. The genotypes of p63^{+/ΔTID} mice were verified by Sanger sequencing (FIGS. 8A and 8B), and very low levels of p63 protein were detected in the ovaries of P1) p63^{+/ΔTID} mice on postnatal day 1 (P1) by Western blot; substantial oocyte loss was observed through immunofluorescence staining (FIG. 8C). No significant abnormalities were observed in adult p63^{+/ΔTID} mice, and no significant difference in body weight was observed compared to WT mice, but malep63^{+/ΔTID} mice were observed to be fertile, while females were observed to be completely sterile (FIG. 8D).

(2) p63^{+/ΔTID} Female Mice Exhibited POI-Like Phenotype

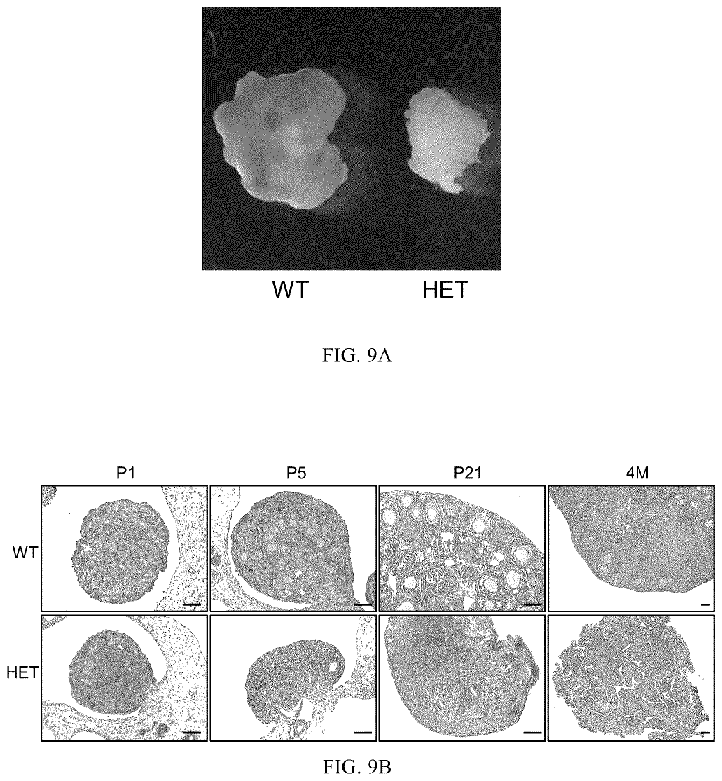

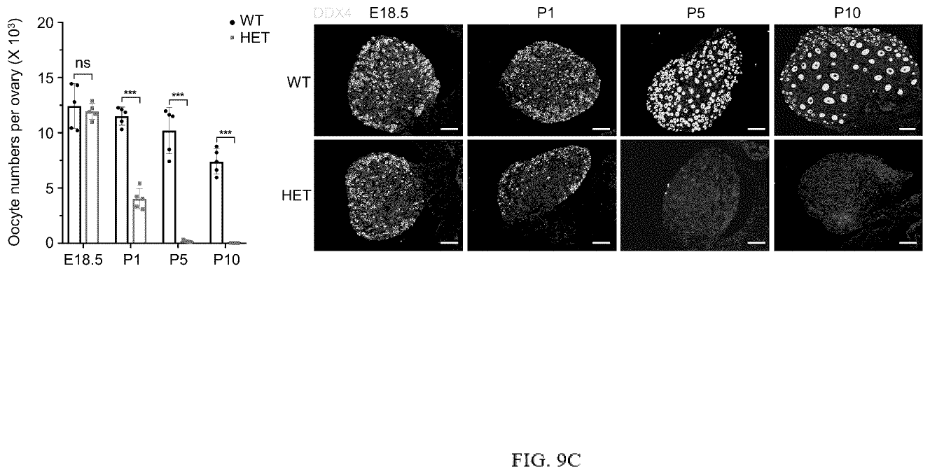

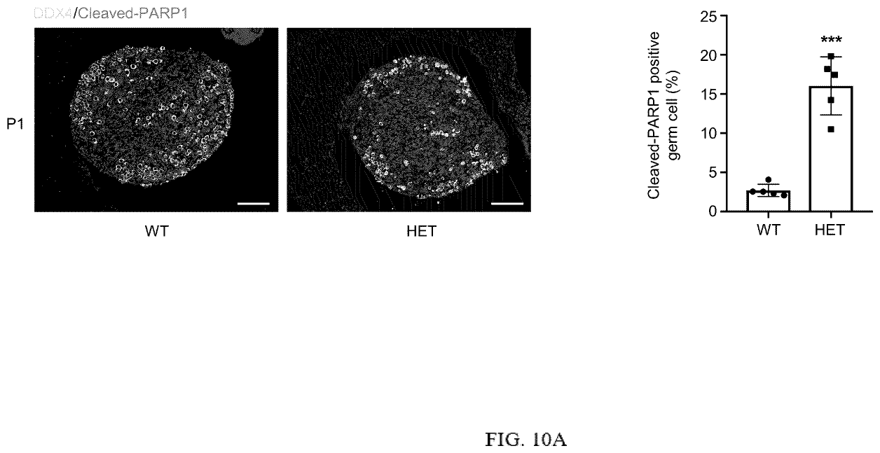

To identify the cause of female sterility in p63^{+/ΔTID}, a detailed analysis of ovarian tissues was performed. The ovaries of 4-month (4M) p63^{+/ΔTID} were significantly smaller. Further ovarian tissue sectioning for HE staining revealed that follicles decreased significantly at P1, were almost invisible at P5, and completely disappeared at P21 and 4M (FIGS. 9a and 9B). Further immunofluorescence staining revealed that there was no significant difference in the number of oocytes between p63^{+/ΔTID} and WT mice at E18.5, but it decreased to about 40% of that in WT mice at P1, and oocytes in p63^{+/ΔTID} mice were occasionally visible at P5 and completely disappeared at P10 (FIG. 9C). These results suggest that deletion of p63 TID leads to rapid depletion of oocytes and loss of fertility in mice, which is similar to the phenotype of human POI.

(3) Spontaneous Apoptosis Occurred in Oocytes of p63^{+/ΔTID} Mice

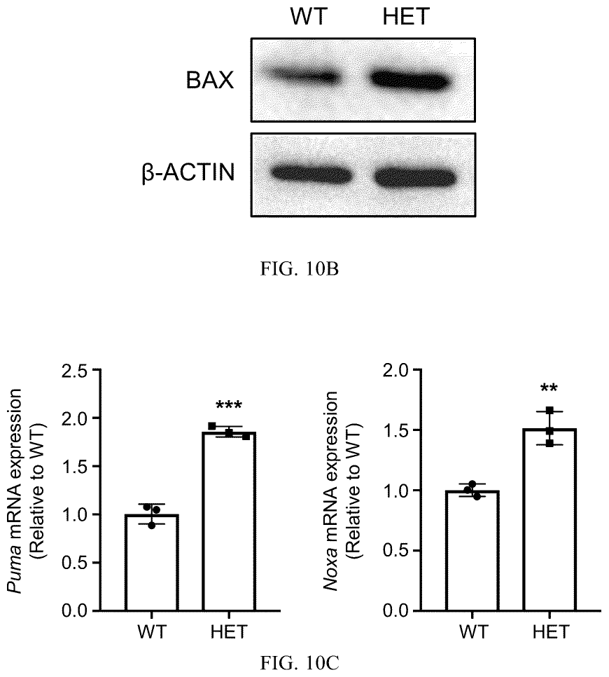

To further investigate the molecular mechanism of rapid oocyte loss in p63^{+/ΔTID} mice, we examined apoptosis in mouse ovaries. Immunofluorescence staining of P1 ovarian sections indicated a significant increase in Cleaved-PARP1 positive oocytes in p63^{+/ΔTID} ovaries (FIG. 10A). Meanwhile, increased expression of BAX protein was also detected in P1 p63^{+/ΔTID} ovaries, and the mRNA levels of two apoptosis-associated target genes, Puma and Noxa, downstream of p63, also significantly increased (FIGS. 10B and 10C). These results suggest that even in the absence of exogenous damage, the absence of p63 TID is sufficient to induce spontaneous apoptosis in oocytes, ultimately resulting in premature loss of ovarian function in mice.

It should be noted, finally, that the foregoing are merely preferred embodiments of the present invention and are not intended to limit the invention. Despite the detailed explanations of the various embodiments provided earlier, those skilled in the art should recognize that modifications can still be made to the technological solutions described in the aforementioned embodiments, or equivalent substitutions can be made for some of the technical features therein. Any modification, equivalent replacement and improvement, etc. performed following the principles of the present invention shall be covered by the protection of the present invention.

What is claimed is:

1. Application of substances for detecting related genes in the preparation of products for detecting premature ovarian insufficiency;
Said relevant genes include ALOX12, BMP6, CPEB1, H1-8, HMMR, HSD17B1, KASH5, LGR4, MCMDC2, MEIOSIN, MST1R, NUP43, PPM1B, PRDM1, RFWD3, SHOC1, SLX4, STRA8, ZAR1, and ZP3.

2. The application according to claim 1, wherein said substances for detecting relevant genes include substances that detect the expression levels, protein modification status (including methylation, acetylation, phosphorylation, adenylation, and ubiquitination), and mutation status of the genes or proteins encoded by the genes;
Said substances for detecting the aforementioned relevant genes specifically include substances based on mass spectrometry, DNA microarray, sequencing, allele-specific probe hybridization, restriction fragment analysis, oligonucleotide ligation assays, single-strand conformation polymorphism analysis, and allele-specific amplification assays.

3. The application according to claim 1, wherein the specific information about the relevant genes and their mutation sites are as follows:

| Genes | Gene ID | Mutation site |
|---|---|---|
| PPM1B | 5495 | NM_002706.6: c.1253dup |
| ALOX12 | 239 | NM_000697.3: c.1909G > T |
| LGR4 | 55366 | NM_018490.5: c.2449C > T |
| MCMDC2 | 157777 | NM_173518.5: c.204del |
| ALOX12 | 239 | NM_000697.3: c.542 + 1G > A |
| HSD17B1 | 3292 | NM_001330219.3: c.313G > T |
| BMP6 | 654 | NM_001718.6: c.472C > T |
| SHOC1 | 158401 | NM_173521.5: c.3903G > A |
| CCDC155 | 147872 | NM_144688.5: c.1269 + 2T > A |
| SHOC1 | 158401 | NM_173521.5: c.231_232del |
| CCDC155 | 147872 | NM_144688.5: c.218del |
| HMMR | 3161 | NM_001142556.2: c.1386-2A > C |
| RFWD3 | 55159 | NM_001370535.1: c.789del |
| STRA8 | 346673 | NM_182489.1: c.258 + 1G > A |
| LGR4 | 55366 | NM_018490.5: c.903-3_903-2del |
| PRDM1 | 639 | NM_001198.4: c.1866C > G |
| MST1R | 4486 | NM_002447.4: c.1480_1481del |
| ALOX12 | 239 | NM_000697.3: c.805C > T |
| HSD17B1 | 3292 | NM_001330219.3: c.721-1G > C |
| HSD17B1 | 3292 | NM_001330219.3: c.947dup |
| MST1R | 4486 | NM_002447.4: c.3846del |
| NUP43 | 348995 | NM_198887.3: c.641del |
| ZAR1 | 326340 | NM_175619.3: c.1131 + 2T > C |
| SLX4 | 84464 | NM_032444,4: c.100C > T |
| RFWD3 | 55159 | NM_001370535.1: c.1561_1562del |
| SHOC1 | 158401 | NM_173521.5: c.1939 + 2T > A |
| HMMR | 3161 | NM_001142556.2: c.667G > T |
| ZP3 | 7784 | NM_001110354.2: c.52G > T |
| HSD17B1 | 3292 | NM_001330219.3: c.313G > T |
| ZP3 | 7784 | NM_001110354.2: c.1028G > A |
| CPEB1 | 64506 | NM_001365240.1: c.1438_1444del |
| ZP3 | 7784 | NM_001110354.2: c.633_652del |
| SHOC1 | 158401 | NM_173521.5: c.3101_3102dup |
| SLX4 | 84464 | NM_032444.4: c.4390_4393dup |
| ZAR1 | 326340 | NM_175619.3: c.119G > A |
| MEIOSIN | 388553 | NM_001310124.2: c.654del |
| HSD17B1 | 3292 | NM_001330219.3: c.97 + 1G > A |
| CPEB1 | 64506 | NM_001365240.1: c.1140_1144 + 10del |
| ZAR1 | 326340 | NM_175619.3: c.270_297dup |
| ZAR1 | 326340 | NM_175619.3: c.44dup |
| H1FOO | 132243 | NM_153833.2: c.251_261del |
| SLX4 | 84464 | NM_032444,4: c.928C > T |
| ZAR1 | 326340 | NM_175619.3: c.1132-2A > C |
| H1FOO | 132243 | NM_153833.2: c.212del |
| NUP43 | 348995 | NM_198887.3: c.544C > T |
| MCMDC2 | 157777 | NM_173518.5: c.685C > T |
| ZAR1 | 326340 | NM_175619.3: c.270_297dup |
| ZAR1 | 326340 | NM_175619.3: c.1108C > T |
| HMMR | 3161 | NM_001142556.2: c.145 + 1G > A |
| MST1R | 4486 | NM_002447.4: c.3991C > T |
| CCDC155 | 147872 | NM_144688.5: c.876 + 1G > A |
| PPM1B | 5495 | NM_002706.6: c.644_671del |
| PRDM1 | 639 | NM_001198.4: c.2322_2325dup |
| SHOC1 | 158401 | NM_173521.5: c.231_232del |
| BMP6 | 654 | NM_001718.6: c.1141C > T |
| HMMR | 3161 | NM_001142556.2: c.429del |
| MEIOSIN | 388553 | NM_001310124.2: c.214A > T |
| LGR4 | 55366 | NM_018490.5: c.1580-2dup |
| PRDM1 | 639 | NM_001198.4: c.32del |
| MST1R | 4486 | NM_002447.4: c.1230 + 2T > C |
| SHOC1 | 158401 | NM_173521.5: c.143_146del |
| NUP43 | 348995 | NM_198887.3: c.639del. |

4. The application according to claim 1, wherein said product further comprises substances for detecting the following POT pathogenic gene P/LP mutation sites, wherein the information on said POI pathogenic genes and mutation sites is as follows:

| Genes | Gene ID | Mutation site |
|---|---|---|
| GDF9 | 2661 | NM_005260.5: c.612del |
| AARS2 | 57505 | NM_020745.4: c.1360G > T |
| BLM | 641 | NM_001287246.2: c.1949C > T |
| RECQL4 | 9401 | NM_004260.3: c.3034T > C |
| MCM8 | 84515 | NM_001281521.1: c.1553G > A |
| BMP15 | 9210 | NM_005448.2: c.373C > T |
| PMM2 | 5373 | NM_000303.3: c.526G > A |
| FIGLA | 344018 | NM_001004311.3: c.385-2A > G |
| SPIDR | 23514 | NM_001080394.4: c.2102del |
| RECQL4 | 9401 | NM_004260.3: c.2886-1G > A |
| HFM1 | 164045 | NM_001017975.5: c.3698C > T |
| WRN | 7486 | NM_000553.6: c.1270-1G > A |
| RECQL4 | 9401 | NM_004260.3: c.3515A > G |
| MSH4 | 4438 | NM_002440.4: c.911dup |
| HFM1 | 164045 | NM_001017975.5: c.2336A > C |
| GDF9 | 2661 | NM_005260.5: c.333C > G |
| LMNA | 4000 | NM_170707.4: c.991C > T |
| SPIDR | 23514 | NM_001080394.4: c.2209C > T |
| HFM1 | 164045 | NM_001017975.5: c.1792G > C |
| HFM1 | 164045 | NM_001017975.5: c.3784G > A |
| FIGLA | 344018 | NM_001004311.3: c.385-2A > G |
| HSF2BP | 11077 | NM_007031.2: c.291 + 1G > T |
| MRPS22 | 56945 | NM_020191.4: c.1043_1046dup |
| HARS2 | 23438 | NM_001363535.2: c.567del |
| BLM | 641 | NM_001287246.2: c.2980del |
| NR5A1 | 2516 | NM_004959.5: c.1262dup |
| SGO2 | 151246 | NM_152524.6: c.195dup |
| MCM8 | 84515 | NM_001281521.1: c.1857G > A |
| MCM9 | 254394 | NM_017696.2: c.398C > G |
| C14orf39 | 317761 | NM_174978.3: c.1037C > G |
| POF1B | 79983 | NM_001307940.2: c.1438-1G > T |
| BRCA2 | 675 | NM_000059.3: c.7084_7085del |
| C14orf39 | 317761 | NM_174978.3: c.1349dup |
| RECQL4 | 9401 | NM_004260.3: c.2128C > T |
| MCM9 | 254394 | NM_017696.2: c.78_80del |
| MCM9 | 254394 | NM_017696.2: c.1390G > A |
| MCM9 | 254394 | NM_017696.2: c.398C > G |
| MCM9 | 254394 | NM_017696.2: c.1306A > G |
| RECQL4 | 9401 | NM_004260.3: c.2404G > T |
| RECQL4 | 9401 | NM_004260.3: c.2554_2559dup |
| WDR62 | 284403 | NM_001083961.2: c.3203_3206del |
| HFM1 | 164045 | NM_001017975.5: c.3477A > C |
| WDR62 | 284403 | NM_001083961.2: c.178-2A > G |
| COX10 | 1352 | NM_001303.4: c.954_955del |
| HARS2 | 23438 | NM_001363535.2: c.1171_1183del |
| HFM1 | 164045 | NM_001017975.5: c.2028_2029del |
| AARS2 | 57505 | NM_020745.4: c.985del |
| NUP107 | 57122 | NM_020401.4: c.262C > T |
| AARS2 | 57505 | NM_020745.4: c.2005C > T |
| SPIDR | 23514 | NM_001080394.4: c.2656_2660del |
| PSMC3IP | 29893 | NM_016556.4: c.597 + 1G > T |
| FIGLA | 344018 | NM_001004311.3: c.1A > G |
| NR5A1 | 2516 | NM_004959.5: c.821T > C |
| BLM | 641 | NM_001287246.2: c.2310C > G |
| HFM1 | 164045 | NM_001017975.5: c.1730A > G |
| SPIDR | 23514 | NM_001080394.4: c.1174G > T |
| AIRE | 326 | NM_000383.4: c.993del |
| HSF2BP | 11077 | NM_007031.2: c.973G > T |
| BMP15 | 9210 | NM_005448.2: c.542G > A |
| EXO1 | 9156 | NM_130398.4: c.353_354del |
| SPIDR | 23514 | NM_001080394.4: c.1181C > G |
| HFM1 | 164045 | NM_001017975.5: c.3934C > T |
| RECQL4 | 9401 | NM_004260.3: c.3418del |
| BRCA2 | 675 | NM_000059.3: c.1910-1G > A |
| RECQL4 | 9401 | NM_004260.3: c.1918C > G |
| MCM8 | 84515 | NM_001281521.1: c.1111_1112del |
| WDR62 | 284403 | NM_001083961.2: c.1255C > T |
| GALT | 2592 | NM_000155.4: c.551del |
| MCM9 | 254394 | NM_017696.2: c.1151-1G > A |
| MCM9 | 254394 | NM_017696.2: c.322G > T |
| RECQL4 | 9401 | NM_004260.3: c.2233C > T |
| AARS2 | 57505 | NM_020745.4: c.298C > T |
| MCM9 | 254394 | NM_017696.2: c.1390G > A |
| POF1B | 79983 | NM_001307940.2: c.137dup |
| MSH4 | 4438 | NM_002440.4: c.2546A > T |
| CLPP | 8192 | NM_006012.4: c.6G > A |
| STAR | 6770 | NM_000349.3: c.403G > T |
| FANCM | 57697 | NM_020937.4: c.5653C > T |
| GNAS | 2778 | NM_080425.3: c.344G > A |
| FOXL2 | 668 | NM_023067.4: c.427dup |
| TP63 | 8626 | NM_003722.5: c.1703del |
| AIRE | 326 | NM_000383.4: c.1182C > A |
| PMM2 | 5373 | NM_000303.3: c.492del |
| RECQL4 | 9401 | NM_004260.3: c.2288G > A |
| SPATA22 | 84690 | NM_001321337.1: c.400C > T |
| POLG | 5428 | NM_001126131.2: c.1168del |
| SPIDR | 23514 | NM_001080394.4: c.3G > A |
| MSH4 | 4438 | NM_002440.4: c.1345T > G |
| MSH4 | 4438 | NM_002440.4: c.1094T > A |
| AIRE | 326 | NM_000383.4: c.44G > A |
| FANCM | 57697 | NM_020937.4: c.1213C > T |
| ATM | 472 | NM_000051.3: c.5830del |
| BLM | 641 | NM_001287246.2: c.2693G > A |
| BLM | 641 | NM_001287246.2: c.1019A > G |
| HFM1 | 164045 | NM_001017975.5: c.1130A > G |
| BNC1 | 646 | NM_001717.4: c.2476C > T |
| PMM2 | 5373 | NM_000303.3: c.448-2A > G |
| NR5A1 | 2516 | NM_004959.5: c.292A > T |
| STAG3 | 10734 | NM_001282717.1: c.2938del |
| HARS2 | 23438 | NM_001363535.2: c.1171_1183del |
| MCM9 | 254394 | NM_017696.2: c.3160A > C |
| HFM1 | 164045 | NM_001017975.5: c.1153A > G |
| MSH4 | 4438 | NM_002440.4: c.1603C > T |
| MCM8 | 84515 | NM_001281521.1: c.1385C > A |
| MRPS22 | 56945 | NM_020191.4: c.1043_1046dup |
| MCM9 | 254394 | NM_017696.2: c.816T > G |
| NR5A1 | 2516 | NM_004959.5: c.245-2A > G |
| FSHR | 2492 | NM_000145.4: c.447-1G > C |
| HARS2 | 23438 | NM_001363535.2: c.567del |
| CLPP | 8192 | NM_006012.4: c.368-3_368-2dup |
| MSH4 | 4438 | NM_002440.4: c.2179G > C |
| WRN | 7486 | NM_000553.6: c.~76-1G > A |
| SYCP2L | 221711 | NM_001040274.3: c.1219-1G > C |
| POLG | 5428 | NM_001126131.2: c.2677T > C |
| STAR | 6770 | NM_000349.3: c.125del |
| RECQL4 | 9401 | NM_004260.3: c.3062_3079dup |
| FANCL | 55120 | NM_001114636.1: c.555 + 2T > G |
| MCM9 | 254394 | NM_017696.2: c.595T > G |
| ERCC6 | 2074 | NM_000124.4: c.1821 + 1G > T |
| NUP107 | 57122 | NM_020401.4: c.8 + 1G > C. |

5. The application according to claim 1, wherein said product further comprises the detection of TP63 gene mutation sites c.1928G>A, c.1937T>C and c.1964G>A and/or detection of TP63 protein mutation sites p.R643Q, p.L646P, p.R655Q substances.

6. The application according to claim 1, wherein said product is one that can be used for (etiology) screening, (auxiliary) diagnosis, detection, monitoring and predictive evaluation of premature ovarian insufficiency;
Further, said product is primers, probes, nucleic acid membrane strips, (gene or protein) chips, preparations, kits, instruments, detection devices and equipment.

7. An product for detecting premature ovarian insufficiency, wherein said product contains substances for detecting related genes;
Wherein, said substances for detecting related genes are shown in the application described in claim 1.

8. An product for detecting premature ovarian insufficiency, wherein said product contains substances for detecting related genes;
Wherein, said substances for detecting related genes are shown in the application described in claim 3.

9. An product for detecting premature ovarian insufficiency, wherein said product contains substances for detecting related genes;
Wherein, said substances for detecting related genes are shown in the application described in claim 4.

10. An product for detecting premature ovarian insufficiency, wherein said product contains substances for detecting related genes;
Wherein, said substances for detecting related genes are shown in the application described in claim 5.

11. A system for detecting premature ovarian insufficiency, wherein said system comprises:
i) An analysis unit, which comprises: Substances selected from a related genes in the subject's sample to be tested from the application described in claim 1; and
ii) An assessment unit, which comprises: Judging the conditions of the subject based on the profile of the relevant genes identified in i;
Wherein, the test sample may be a human sample, more specifically, said test sample comprises peripheral blood from the subject;
Said subject's disease profile includes screening, (auxiliary) diagnosis, detection, monitoring, and predictive evaluation of premature ovarian insufficiency in the subject.

12. A system for detecting premature ovarian insufficiency, wherein said system comprises:
i) An analysis unit, which comprises: Substances selected from a related genes in the subject's sample to be tested from the application described in claim 2; and
ii) An assessment unit, which comprises: Judging the conditions of the subject based on the profile of the relevant genes identified in i;
Wherein, the test sample may be a human sample, more specifically, said test sample comprises peripheral blood from the subject;
Said subject's disease profile includes screening, (auxiliary) diagnosis, detection, monitoring, and predictive evaluation of premature ovarian insufficiency in the subject.

13. A system for detecting premature ovarian insufficiency, wherein said system comprises:
i) An analysis unit, which comprises: Substances selected from a related genes in the subject's sample to be tested from the application described in claim 3; and
ii) An assessment unit, which comprises: Judging the conditions of the subject based on the profile of the relevant genes identified in i;
Wherein, the test sample may be a human sample, more specifically, said test sample comprises peripheral blood from the subject;
Said subject's disease profile includes screening, (auxiliary) diagnosis, detection, monitoring, and predictive evaluation of premature ovarian insufficiency in the subject.

14. A system for detecting premature ovarian insufficiency, wherein said system comprises:
i) An analysis unit, which comprises: Substances selected from a related genes in the subject's sample to be tested from the application described in claim 4; and
ii) An assessment unit, which comprises: Judging the conditions of the subject based on the profile of the relevant genes identified in i;
Wherein, the test sample may be a human sample, more specifically, said test sample comprises peripheral blood from the subject;
Said subject's disease profile includes screening, (auxiliary) diagnosis, detection, monitoring, and predictive evaluation of premature ovarian insufficiency in the subject.

15. A system for detecting premature ovarian insufficiency, wherein said system comprises:
i) An analysis unit, which comprises: Substances selected from a related genes in the subject's sample to be tested from the application described in claim 5; and
ii) An assessment unit, which comprises: Judging the conditions of the subject based on the profile of the relevant genes identified in i;
Wherein, the test sample may be a human sample, more specifically, said test sample comprises peripheral blood from the subject;
Said subject's disease profile includes screening, (auxiliary) diagnosis, detection, monitoring, and predictive evaluation of premature ovarian insufficiency in the subject.

16. A system for detecting premature ovarian insufficiency, wherein said system comprises:
i) An analysis unit, which comprises: Substances selected from a related genes in the subject's sample to be tested from the application described in claim 6; and
ii) An assessment unit, which comprises: Judging the conditions of the subject based on the profile of the relevant genes identified in i;
Wherein, the test sample may be a human sample, more specifically, said test sample comprises peripheral blood from the subject;
Said subject's disease profile includes screening, (auxiliary) diagnosis, detection, monitoring, and predictive evaluation of premature ovarian insufficiency in the subject.

17. An application of the relevant genes in preparing drugs for premature ovarian insufficiency and/or screening drugs for premature ovarian insufficiency;
Said drugs for premature ovarian insufficiency refer to the ones that prevent and/or treat premature ovarian insufficiency;
Wherein, said relevant genes are shown in the application described in claim 1.

18. An application of the relevant genes in preparing drugs for premature ovarian insufficiency and/or screening drugs for premature ovarian insufficiency;
Said drugs for premature ovarian insufficiency refer to the ones that prevent and/or treat premature ovarian insufficiency;
Wherein, said relevant genes are shown in the application described in claim 3.

19. An application of the relevant genes in preparing drugs for premature ovarian insufficiency and/or screening drugs for premature ovarian insufficiency;
Said drugs for premature ovarian insufficiency refer to the ones that prevent and/or treat premature ovarian insufficiency;
Wherein, said relevant genes are shown in the application described in claim 4.

20. An application of the relevant genes in preparing drugs for premature ovarian insufficiency and/or screening drugs for premature ovarian insufficiency;
Said drugs for premature ovarian insufficiency refer to the ones that prevent and/or treat premature ovarian insufficiency;
Wherein, said relevant genes are shown in the application described in claim 5.
